SEQUENCE LISTING (6 sequences)

SEQ ID NO: 1 - protein, 907 aa, synthetic construct
VGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGGSMAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLARPMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGGSGMTEQQWNFAGIEAAASAIQGNVTSIHSLLDEGKQSLTKLAAAWGGSGSEAYQGVQQKWDATATELNNALQNLARTISEAGQAMASTEGNVTGMFAGSSGGSSGMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGSSGGSSGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 2 - protein, 1184 aa, synthetic construct
VGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGGSMAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAMTINYQFGDVDAHGAMIRAQAASLEAEHQAIVRDVLAAGDFWGGAGSVACQEFITQLGRNFQVIYEQANAHGQKVQAAGNNMAQTDSAVGSSWAGGSGMLWHAMPPELNTARLMAGAGPAPMLAAAAGWQTLSAALDAQAVELTARLNSLGEAWTGGGSDKALAAATPMVVWLQTASTQAKTRAMQATAQAAAYTQAMATTPSLPEIAANHITQAVLTATNFFGINTIPIALTEMDYFIRMWNQAALAMEVYQAETAVNTLFEKLEPMASILDPGASQSTTNPIFGMPSPGSSTPVGQLPPAATQTLGQLGEMSGPMQQLTQPLQQVTSLFSQVGGTGGGNPADEEAAQMGLLGTSPLSNHPLAGGSGPSAGAGLLRAESLPGAGGSLTRTPLMSQLIEKPVAPSVMPAAAAGSSATGGAAPVGAGAMGQGAQSGGSTRPGLVAPAPLAQEREEDDEDDWDEEDDWGSSGGSSGAMSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGSSGGSSGAMDPVDAVINTTCNYGQVVAALNATDPGAAAQFNASPVAQSYLRNFLAAPPPQRAAMAAQLQAVPGAAQYIGLVESVAGSCNNY

SEQ ID NO: 3 - protein, 4 aa, synthetic construct
GGSG

SEQ ID NO: 4 - protein, 8 aa, synthetic construct
GSSGGSSG

SEQ ID NO: 5 - protein, 100 aa, synthetic construct
VGLNRFMRAMMVVFITANCITINPDIIFAATDSEDSSLNTDEWEEEKTEEQPSEVNTGPRYETAREVSSRDIEELEKSNKVKNTNKADLIAMLKAKAEKG

SEQ ID NO: 6 - protein, 4 aa, synthetic construct
GSSG

(19) United States
(12) Patent Application Publication
(10) Pub. No.: US 2025/0152690 A1
(43) Pub. Date: May 15, 2025

(54) NOVEL LIVE MULTI-ANTIGENIC RECOMBINANT VACCINE AGAINST TUBERCULOSIS

(71) Applicant: THE REGENTS OF THE UNIVERSITY OF CALIFORNIA, Oakland, CA (US)

(72) Inventors: Marcus A. Horwitz, Los Angeles, CA (US); Qingmei Jia, Los Angeles, CA (US)

(73) Assignee: The Regents of the University of California, Oakland, CA (US)

(21) Appl. No.: 18/836,272

(22) PCT Filed: Feb. 16, 2023
(86) PCT No.: PCT/US23/62733

§ 371 Date: Aug. 6, 2024

Related U.S. Application Data
(60) Provisional application No. 63/312,289, filed on Feb. 21, 2022.

(51) Int. Cl. A61K 39/04; A61K 9/00; A61K 39/00; A61P 31/06

(52) U.S. Cl. CPC A61K 39/04; A61K 9/0019; A61P 31/06; A61K 2039/522; A61K 2039/523; A61K 2039/54; A61K 2039/572

(57) ABSTRACT

Tuberculosis (TB), caused by Mycobacterium tuberculosis (Mtb), remains a deadly global disease. Embodiments of the invention comprise an improved vaccine for generating an immune response and preventing or treating mycobacterial diseases such as tuberculosis in humans and animals. Embodiments of the invention also comprise a method for using the vaccine against such mycobacterial diseases.

### CROSS-REFERENCE TO RELATED APPLICATIONS

This application claims the benefit under 35 U.S.C. Section 119 (e) of co-pending and commonly-assigned U.S. Provisional Patent Application Ser. No. 63/312,289, filed on Feb. 21, 2022, and entitled “NOVEL LIVE MULTI-ANTIGENIC RECOMBINANT VACCINE AGAINST TUBERCULOSIS” which application is incorporated by reference herein. This application is related to PCT International Publication No. WO 2011/159814, the contents of which are incorporated by reference.

### STATEMENT OF GOVERNMENT SUPPORT

This invention was made with government support under Grant Number AI135631, awarded by the National Institutes of Health. The government has certain rights in the invention.

### 1. FIELD OF THE INVENTION

The present invention relates to methods and compositions of matter that are useful for preventing or reducing the possibility of infection caused by Mycobacterium tuberculosis, the agent of tuberculosis, and infection by other pathogenic strains of mycobacteria in humans and/or animals including Mycobacterium bovis and Mycobacterium leprae.

### 2. BACKGROUND

Tuberculosis (TB), caused by Mycobacterium tuberculosis (Mtb), remains a deadly global disease. It is estimated that one quarter of the world's population has been infected with Mtb, most of whom develop latent TB infection, and that 10 million people develop active TB and 1.5 million people die of TB annually. Mycobacterium bovis Bacillus Calmette-Guérin (BCG), developed more than 100 years ago and the only licensed vaccine against TB, has been used to vaccinate infants and to protect young children against severe forms of TB; however BCG has shown variable efficacy in preventing pulmonary TB in adolescents and adults, the most prevalent form (1). As BCG has been widely used worldwide to vaccinate 88% of infants within the first year of their life (1, 2), booster vaccines that improve upon the efficacy of BCG, even to a small extent, could have a significant impact on the TB pandemic.

Several strategies have been employed to develop booster vaccines for TB, primarily protein/adjuvant vaccines comprising fusion proteins of selected Mtb antigens administered with a strong T-cell stimulating adjuvant and viral-vectored vaccines, wherein viruses including adenovirus, Modified Vaccinia Ankara virus, and cytomegalovirus express recombinant proteins (3-9). In contrast, we have employed a highly attenuated replicating bacterium as a vaccine vector, Lm ΔactA ΔinlB prfA*, a Listeria monocytogenes with deletions in two major virulence genes (actA and inlB) and a single amino acid substitution (G155S) in PrfA (positive regulatory factor A) resulting in constitutive overexpression of PrfA and PrfA-dependent genes, a modification exploited to enhanced vaccine efficacy (10). Listeria monocytogenes is an intracellular bacterium that invades mononuclear phagocytes, resides in a membrane-bound phagosome, and ultimately escapes the phagosome to reside and multiply in the cytoplasm (11). Its intraphagosomal and intracytoplasmic locations favor antigen presentation via both MHC class I and II, respectively, allowing induction of both CD4+ and CD8+ antigen-specific T cells, both important to immunity against TB. A Listeria vector also has other immunologic advantages including the capacity to carry and express a large amount of recombinant protein cargo; the ability to disseminate to organs that are impacted by Mtb, such as the lung and spleen, before being cleared by the immune system, thereby promoting local immunity at sites of Mtb infection; and the fact that pre-existing immunity does not negatively affect efficacy (12, 13). Additional practical advantages of a Listeria vectored vaccine are an established safety profile (14), as the vector has been used safely in cancer vaccines, and low cost of manufacture in simple broth culture, without the need for extensive purification as in the case of protein/adjuvant and viral-vectored vaccines.

A safe and effective vaccine against M. tuberculosis or other species of the genus Mycobacterium that is superior to the currently available vaccines is sorely needed. There is also a need for a M. tuberculosis booster vaccine or a vaccine that can improve the potency of the currently available vaccines by even a small amount. The disclosure provided herein meets this need.

### SUMMARY OF THE INVENTION

The present disclosure provides a vaccine and method for preventing, reducing the possibility of or treating tuberculosis in humans and animals that is better than the current commercially available vaccines and methods in protecting against pulmonary tuberculosis and dissemination of bacteria to the spleen and other organs. The present disclosure also provides a vaccine and method for preventing, reducing the possibility of or treating leprosy and other mycobacterial diseases. Moreover, the present disclosure provides a vaccine that is easier and cheaper to manufacture than both virus-vectored vaccines, which must be grown in tissue culture cells and then purified, and protein-in-adjuvant vaccines, where the protein needs to be purified. As noted below, the M. tuberculosis vector vaccine described in the present disclosure can simply be grown in broth culture—no purification is necessary.

Listeria monocytogenes is an intracellular bacterium that invades mononuclear phagocytes, resides in a membrane-bound phagosome, and ultimately escapes the phagosome to reside and multiply in the cytoplasm. Its intraphagosomal and intracytoplasmic locations favor antigen presentation via both MHC class I and II, respectively, allowing induction of both CD4+ and CD8+ antigen-specific T cells, both important to immunity against TB. A Listeria vector also has other immunologic advantages including the capacity to carry and express a large amount of recombinant protein cargo; the ability to disseminate to organs that are impacted by M. tuberculosis, such as the lung and spleen, before being cleared by the immune system, thereby promoting local immunity at sites of Mtb infection; and the fact that pre-existing immunity does not negatively affect efficacy. Additional practical advantages of a Listeria vectored vaccine are an established safety profile, as the vector has been used safely in cancer vaccines, and low cost of manufacture in simple broth culture, without the need for extensive purification as in the case of protein/adjuvant and viral-vectored vaccines. Protein/adjuvant vaccines and non-replicating virus-vectored vaccines lack many of these advantages.

Embodiments of the invention include the aforementioned Listeria monocytogenes ΔactA ΔinlB prfA* vector into which antigen cassettes comprising all or parts of various key immunoprotective antigens of M. tuberculosis have been inserted. Each vaccine is administered intradermally or by another route, e.g. subcutaneously, intramuscularly, intranasally, inhaled, or even orally to a mammalian host. The vaccine can be administered as part of a homologous or heterologous prime-boost vaccination strategy. The vaccine induces a strong cell-mediated immune response to pathogen antigens in the vaccine including both CD4 and CD8 T cells.

The invention disclosed herein has a number of embodiments. Embodiments of the invention include compositions of matter comprising at least one fusion protein having antigenic epitopes present in at least five Mycobacterium tuberculosis proteins selected from: immunogenic protein MPT64 (“23.5/Mpt64”), ESAT-6-like protein EsxH (“TB10.4/EsxH”), 6 kDa early secretory antigenic target (“ESAT6/EsxA”), ESAT-6-like protein EsxB (“CFP10/EsxB”), and diacylglycerol acyltransferase/mycolyltransferase Ag85B (“r30/Antigen 85B”); ESAT-6-like protein EsxN (“EsxN”); PPE family immunomodulator PPE68 (“PPE68”); ESX-1 secretion-associated protein EspA (“EspA”) and low molecular weight T-cell antigen TB8.4 (“TB8.4”), wherein the composition comprises live attenuated Listeria monocytogenes expressing the at least one fusion protein. Embodiments of the invention further include polynucleotides encoding the fusion proteins disclosed herein, for example such polynucleotides disposed within a vector.

Typically with the live attenuated Listeria monocytogenes compositions expressing the fusion proteins disclosed herein, when administered to mice as a vaccine, the composition elicits an immune response to Mycobacterium tuberculosis exposure in the mice characterized by an at least 10% reduction in Mycobacterium tuberculosis colony forming units in lungs of mice administered the vaccine as compared to lungs of control mice administered a control composition lacking antigenic epitopes present in Mycobacterium tuberculosis. In illustrative working embodiments of the invention, the fusion protein comprises antigenic epitopes present in the polypeptide sequence:

(SEQ ID NO: 1)
VGLNRFMRAMMVVFITANCITINPDIIFAATDSEDSSLNTDEWEEEKTE
EQPSEVNTGPRYETAREVSSRDIEELEKSNKVKNTNKADLIAMLKAKAE
KGGSMAPKTYCEELKGTDTGQACQIQMSDPAYNINISLPSYYPDQKSLE
NYIAQTRDKFLSAATSSTPREAPYELNITSATYQSAIPPRGTQAVVLKV
YQNAGGTHPTTTYKAFDWDQAYRKPITYDTLWQADTDPLPVVFPIVQGE
LSKQTGQQVSIAPNAGLDPVNYQNFAVTNDGVIFFFNPGELLPEAAGPT
QVLVPRSAIDSMLARPMSQIMYNYPAMLGHAGDMAGYAGTLQSLGAEIA
VEQAALQSAWQGDTGITYQAWQAQWNQAMEDLVRAYHAMSSTHEANTMA
MMARDTAEAAKWGGGGSGMTEQQWNFAGIEAAASAIQGNVTSIHSLLDE
GKQSLTKLAAAWGGSGSEAYQGVQQKWDATATELNNALQNLARTISEAG
QAMASTEGNVTGMFAGSSGGSSGMAEMKTDAATLAQEAGNFERISGDLK
TQIDQVESTAGSLQGQWRGAAGTAAQAAVVRFQEAANKQKQELDEISTN
IRQAGVQYSRADEEQQQALSSQMGFGSSGGSSGAFSRPGLPVEYLQVPS
PSMGRDIKVQFQSGGNNSPAVYLLDGLRAQDDYNGWDINTPAFEWYYQS
GLSIVMPVGGQSSFYSDWYSPACGKAGCQTYKWETFLTSELPQWLSANR
AVKPTGSAAIGLSMAGSSAMILAAYHPQQFIYAGSLSALLDPSQGMGPS
LIGLAMGDAGGYKAADMWGPSSDPAWERNDPTQQIPKLVANNTRLWVYC
GNGTPNELGGANIPAEFLENFVRSSNLKFQDAYNAAGGHNAVFNFPPNG
THSWEYWGAQLNAMKGDLQSSLGAG;

and/or
the fusion protein comprises antigenic epitopes present in the polypeptide sequence:

(SEQ ID NO: 2)
VGLNRFMRAMMVVFITANCITINPDIIFAATDSEDSSLNTDEWEEEKTE
EQPSEVNTGPRYETAREVSSRDIEELEKSNKVKNTNKADLIAMLKAKAE
KGGSMAPKTYCEELKGTDTGQACQIQMSDPAYNINISLPSYYPDQKSLE
NYIAQTRDKFLSAATSSTPREAPYELNITSATYQSAIPPRGTQAVVLKV
YQNAGGTHPTTTYKAFDWDQAYRKPITYDTLWQADTDPLPVVFPIVQGE
LSKQTGQQVSIAPNAGLDPVNYQNFAVTNDGVIFFFNPGELLPEAAGPT
QVLVPRSAIDSMLAMTINYQFGDVDAHGAMIRAQAASLEAEHQAIVRDV
LAAGDFWGGAGSVACQEFITQLGRNFQVIYEQANAHGQKVQAAGNNMAQ
TDSAVGSSWAGGSGMLWHAMPPELNTARLMAGAGPAPMLAAAAGWQTLS
AALDAQAVELTARLNSLGEAWTGGGSDKALAAATPMVVWLQTASTQAKT
RAMQATAQAAAYTQAMATTPSLPEIAANHITQAVLTATNFFGINTIPIA
LTEMDYFIRMWNQAALAMEVYQAETAVNTLFEKLEPMASILDPGASQST
TNPIFGMPSPGSSTPVGQLPPAATQTLGQLGEMSGPMQQLTQPLQQVTS
LFSQVGGTGGGNPADEEAAQMGLLGTSPLSNHPLAGGSGPSAGAGLLRA
ESLPGAGGSLTRTPLMSQLIEKPVAPSVMPAAAAGSSATGGAAPVGAGA
MGQGAQSGGSTRPGLVAPAPLAQEREEDDEDDWDEEDDWGSSGGSSGAM
SRAFIIDPTISAIDGLYDLLGIGIPNQGGILYSSLEYFEKALEELAAAF
PGDGWLGSAADKYAGKNRNHVNFFQELADLDRQLISLIHDQANAVQTTR
DILEGAKKGLEGEVWEFITNALNGLKELWDKLTGWVTGLFSRGWSNLES
FFAGVPGLTGATSGLSQVTGLFGAAGLSASSGLAHADSLASSASLPALA
GIGGGSGFGGLPSLAQVHAASTRQALRPRADGPVGAAAEQVGGQSQLVS
AQGSQGMGGPVGMGGMHPSSGASKGTTTKKYSEGAAAGTEDAERAPVEA
DAGGGQKVLVRNVVGSSGGSSGAMDPVDAVINTTCNYGQVVAALNATDP
GAAAQFNASPVAQSYLRNFLAAPPPQRAAMAAQLQAVPGAAQYIGLVES
VAGSCNNY.

In certain embodiments of the invention, the composition comprises at least two fusion proteins. For example, in some embodiments of the invention, the Listeria monocytogenes expresses a first fusion protein encoded by a polynucleotide present in a first locus of the Listeria monocytogenes genome and a second fusion protein encoded by a polynucleotide present in a second locus of the Listeria monocytogenes genome. In certain embodiments of the invention, a first fusion protein comprises epitopes present in at least two Mycobacterium tuberculosis proteins selected from: 23.5/Mpt64, TB10.4/EsxH, ESAT6/EsxA, CFP10/EsxB, and r30/Antigen 85B; and a second fusion protein comprises epitopes present in at least two Mycobacterium tuberculosis proteins selected from EsxN; PPE68; EspA and TB8.4.

Embodiments of the invention also include methods of generating an immune response to a Mycobacterium tuberculosis in a mammal (e.g., a mouse, guinea pig or human) comprising administering to the mammal a composition comprising a live attenuated Listeria monocytogenes expressing one or more fusion proteins disclosed herein such that an immune response to Mycobacterium tuberculosis is generated. Typically in such embodiments of the invention, when administered to mice as a vaccine, the composition elicits an immune response to Mycobacterium tuberculosis exposure in the mice characterized by an at least 10%, 25% or 50% (or 0.05, 0.1, or 0.3 log) reduction in Mycobacterium tuberculosis colony forming units in lungs of mice administered the vaccine as compared to lungs of control mice administered a control composition lacking antigenic epitopes present in Mycobacterium tuberculosis.

Typically, the mammal is immunized intranasally, subcutaneously, intradermally, intramuscularly or orally. Certain of these methodological embodiments of the invention include the steps of exposing the mammal to Mycobacterium tuberculosis antigens using a different immunogenic platform administered to the mammal at the same or at a different time, for example methods which comprise immunizing the mammal a composition disclosed herein in combination with Mycobacterium bovis strain Bacille Calmette-Guérin (BCG).

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

Referring now to the drawings and figures:

FIG. 1. Efficacy against Mtb aerosol challenge of boosting BCG-primed mice with a combination of rLm vaccine candidates expressing 4 Mtb antigens Female C57BL/6 mice (n=8/group) were immunized intranasally (i.n.) with PBS (Sham) or BCG at Week 0; not boosted or boosted i.n. twice with 10⁶ CFU total of combined rLm expressing 4 Mtb proteins—rLm3Ag (rLm30-10.4-ESAT6) and rLm23.5 at Weeks 7 and 10; challenged with aerosolized Mtb at Week 13; and euthanized at Week 19 (top panel). Lungs (bottom left panel) and spleens (bottom right panel) of mice were assayed for organ bacterial burden. One symbol represents one animal. Values are means±SEM. ***, P<0.001 by one-way ANOVA; #, P<0.05 by t-test (Prism).

FIG. 2. Efficacy against Mtb aerosol challenge of boosting BCG-primed mice with a combination of rLm vaccine candidates expressing 5 Mtb antigens. a. Efficacy of boosting with 5Ag rLm vaccines i.m. once vs. twice. BALB/cJ mice (n=8/group) were immunized i.d. with PBS or with BCG at Week 0. BCG-primed mice were either not boosted or boosted intramuscularly (i.m.) or subcutaneously (s.q.) once (x1) at Week 18 with Lm vector (LmV) or the combination of an rLm vaccine expressing Mtb fusion protein 23.5-TB10.4-ESAT6-CFP10 (rLm4Ag) and rLm30. An additional group was boosted twice (x2) at Weeks 14 and 18 with rLm4Ag+rLm30. The mice were then challenged with aerosolized Mtb Erdman (average of 19 CFU delivered to the lungs of each animal, as assayed at Day 1 post challenge to 2 mice) at Week 22 and euthanized at Week 32 (top panel). Lungs (bottom left panel) and spleens (bottom right panel) of mice were assayed for organ bacterial burden. Shown are means±SEM. One symbol represents one animal. Organ CFUs were analyzed by One-Way ANOVA with Dunnett's multiple comparisons (Prism 8.02). *, P<0.05; ****, P<0.0001. b. Efficacy of 5Ag rLm vaccines by route of administration. BALB/c mice (8/group) were immunized i.d. with PBS (Sham) or 5×10⁵ CFU BCG at Week 0. Mice immunized i.d. with BCG were either not boosted or boosted i.m., i.d., subcutaneously (s.q.) or intravenously (i.v.) once at Week 18 with 2×10⁶ CFU of the combination of an rLm vaccine expressing Mtb fusion protein 23.5-TB10.4-ESAT6-CFP10 (rLm4Ag) and rLm30. The mice were then challenged at Week 22 with aerosolized Mtb Erdman at the same time as the companion experiment shown in a (average of 19 CFU delivered to the lungs of each animal) and euthanized at Week 26 (top panel). Afterwards, lungs (bottom left panel) and spleens (bottom right panel) were removed and assayed for bacillus burdens. Shown are means±SEM. Each symbol represents one mouse. *, P<0.05 and ****, P<0.0001 by unpaired t-test (Prism).

FIG. 3. Expression of fusion proteins comprising 5 Mtb antigens by 11 new rLm vaccine candidates grown in broth. Glycerol stocks of the Lm vector and each of 11 new rLm vaccine candidates expressing 5 Mtb antigens were grown in Brain Heart Infusion medium supplemented with Streptomycin (200 μg/ml) overnight at 37° C. Cells were collected by centrifugation, lysed in SDS buffer, and the cell lysate processed by standard SDS-PAGE and Western blotting analysis using polyclonal antibody to ActA AK18. In both panels, the expected major protein bands are indicated by red asterisks to the right of each protein band. The Mw of the protein standards are labeled on the left of each panel. The Mtb fusion protein expression cassette and the estimated Mw of the fusion protein for each of the vaccines listed at the top of the gels are described in Table 3. Note: Lane 13 shows one clone and lanes 17 and18 show two different clones of the same construct, expressing the secreted (74 kDa) and non-secreted (78 kDa) fusion proteins.

FIG. 4. Efficacy against Mtb aerosol challenge of priming mice with BCG and boosting them with rLm expressing 5 Mtb antigens. a. Experimental schedule. BALB/c mice (8 or 12/group) were immunized i.d. with PBS (Sham) or 5×10⁵ CFU of BCG at Week 0. Mice immunized i.d. with BCG were either not boosted or boosted once at Week 18 or twice at Weeks 14 and 18 (Group G2 only) with 2×10⁶ CFU of rLm5Ag vaccine candidates. At Week 22, the mice were challenged with aerosolized Mtb Erdman strain (average of 19 CFU delivered to the lungs of each animal, as assayed at Day 1 post challenge to mice in Group A*) and at Week 32, 10 weeks post-challenge, mice were euthanized. b. Organ bacterial burden. Lungs (top) and spleens (bottom) of mice in the vaccinated and challenge groups (described in a Table to the right of the graphs) were removed and assayed for bacillus burdens. Each symbol represents one mouse and the means±SEM are shown as bars. Group designations are listed beneath the horizontal axis. The pink color boxes indicate Group B-BCG prime only. The differences in lung and spleen CFUs between sham group (A) and all other groups were statistically significant (P<0.05-0.0001) (not shown). *, P<0.05, ***, P<0.001, and ****, P<0.0001 vs. Group B (BCG i.d.) by ordinary one-way ANOVA with Dunnett's multiple comparisons test.

FIG. 5. Frequency of cytokine-expressing CD4+ T cells in the lungs and spleens of C57BL/6 primed with BCG and boosted twice with Lm vector or rLm5Ag (30). Mice (n=4/group) were primed with BCG at Week 0 and boosted twice at Weeks 14 and 18 with Lm vector (black bars and symbols) or rLm5Ag (30) (pink bars and symbols) expressing 23.5-TB10.4-ESAT6-CFP10-r30. Six days after the last immunization, mice were euthanized; their lungs and spleens removed; single cell suspensions prepared and stimulated with recombinant proteins 23.5/Mpt64 (a, i), TB10.4/EsxH (b, j), ESAT6/EsxA (c, k), CFP10/EsxB (d, l), r30/Ag85B (e, m), pool of the 5 Ags (f, n), PPD (g, o), or PMA (positive control) (h, p) in the presence of anti-CD28 monoclonal antibody for 6 h [except in the case of PMA (positive control) for 4 h], and the cells assayed by intracellular cytokine staining (ICS) for surface markers of CD4 and intracellular markers of IFN-γ, TNF-α, IL-2, and IL-17A, as indicated below each panel. Each symbol represents one animal. Values are the mean±SEM. *, P<0.05; **, P<0.01; ***, P<0.001; and ****, P<0.0001 by two-way ANOVA with Sidak's post multiple comparisons test.

FIG. 6. Frequency of polyfunctional cytokine-expressing CD4+ T cells in the lungs and spleens of C57BL/6 mice primed with BCG and boosted twice with Lm vector or rLm5Ag (30). Mice were immunized and spleen and lung cells prepared and stimulated with recombinant proteins as described in the legend to FIG. 5. The cells were assayed by ICS for surface markers of CD4 and intracellular markers of IFN-γ, TNF-α, IL-2, and IL-17A. The frequencies of CD4+ T cells expressing 1 or combinations of 2, 3 or 4 of the four cytokines assayed in response to stimulation with r30/Ag85B (a, e), pool of the 5 Ags (b, f), PPD (c, g), or PMA (positive control) (d, h), are shown, as indicated below each panel. Values are the mean±SEM. *, P<0.05; **, P<0.01; and ****, P<0.0001 by two-way ANOVA with Sidak's post multiple comparisons test.

FIG. 7. Frequency of cytokine-expressing CD8+ T cells in the lungs and spleens of C57BL/6 mice primed with BCG and boosted twice with Lm vector or rLm5Ag (30). Mice were immunized and spleen cells prepared and stimulated with recombinant proteins as described in the legend to FIG. 5. The cells were assayed by ICS for surface markers of CD8 and intracellular markers of IFN-γ, TNF-α, IL-2, and IL-17A. The frequencies of CD8+ T cells expressing IFN-γ, TNF-α, IL-2, and IL-17A in response to stimulation with recombinant proteins 23.5/Mpt64 (a, i), TB10.4/EsxH (b, j), ESAT6/EsxA (c, k), CFP10/EsxB (d, l), r30/Ag85B (e, m), pool of the 5 Ags (f, n), PPD (g, o), and PMA (positive control) (h, p) are shown. Each symbol represents one animal. Black bars and symbols: primed-boosted with BCG-LmVector; pink bars and symbols: primed-boosted with BCG-rLm5Ag (30). Values are the mean±SEM. *, P<0.05; ***, P<0.001; and ****, P<0.0001 by two-way ANOVA with Sidak's post multiple comparisons test.

FIG. 8. Frequency of cytokine-expressing CD4+ T cells in the lungs and spleens of BALB/c primed with BCG and boosted twice with Lm vector or rLm5Ag (30). BALB/c mice (n=4/group) were primed with BCG at Week 0 and boosted twice at Weeks 14 and 18 with Lm vector (black bars and symbols) or rLm5Ag (30) (pink bars and symbols) expressing 23.5-TB10.4-ESAT6-CFP10-r30. Six days after the last immunization, mice were euthanized; their spleens and lungs removed; single cell suspensions prepared and stimulated with recombinant proteins 23.5/Mpt64 (a, i), TB10.4/EsxH (b, j), ESAT6/EsxA (c, k), and CFP10/EsxB (d, l), r30/Ag85B (e, m), pool of the 5 Ags (f, n), PPD (g, o), or PMA (positive control) (h, p), and in the presence of anti-CD28 monoclonal antibody for 6 h [except in the case of PMA (positive control) for 4 h], and the cells assayed by intracellular cytokine staining (ICS) for surface markers of CD4 and intracellular markers of IFN-γ, TNF-α, IL-2, and IL-17A, as indicated below each panel. Each symbol represents one animal. Values are the mean±SEM. **, P<0.01 by two-way ANOVA with Sidak's post multiple comparisons test.

FIG. 9. Frequency of cytokine-expressing CD8+ T cells in the lungs and spleens of BALB/c primed with BCG and boosted twice with Lm vector or rLm5Ag (30). BALB/c mice were immunized and spleen and lung cells prepared and stimulated with recombinant proteins as described in the legend to FIG. 8. The cells were assayed by ICS for surface markers of CD8 and intracellular markers of IFN-γ, TNF-α, IL-2, and IL-17A. The frequencies of CD8+ T cells expressing IFN-γ, TNF-α, IL-2, and IL-17A in response to stimulation with recombinant proteins 23.5/Mpt64 (a, i), TB10.4/EsxH (b, j), ESAT6/EsxA (c, k), and CFP10/EsxB (d, l), r30/Ag85B (e, m), pool of the 5 Ags (f, n), PPD (g, o), or PMA (positive control) (h, p) are shown. Each symbol represents one animal. Black bars and symbols: primed-boosted with BCG-LmVector; pink bars and symbols: primed-boosted with BCG-rLm5Ag (30). Values are the mean±SEM. *, P<0.05 and **, P<0.01 by two-way ANOVA with Sidak's post multiple comparisons test.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. As appropriate, procedures involving the use of commercially available kits and reagents are generally carried out in accordance with manufacturer defined protocols and/or parameters unless otherwise noted.

In the following description of the typical embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

While medical practitioners have been working to generate an effective vaccine for tuberculosis for many years, this goal remains elusive. One of the challenges in this area of technology involves determining which M. tuberculosis protein antigen(s) can be used to create effective vaccines (as the Mycobacterium tuberculosis genome comprises around 4.4 million base pairs, and contains around 4,000 genes). Previously we developed a recombinant Listeria monocytogenes ΔactA ΔinlB prfA* vectored vaccine candidate (rLm30) expressing the Mtb 30-kDa major secretory protein (r30/Ag85B/Rv1886) driven by the hly promoter and leader sequence or the actA promoter and leader sequence to facilitate the expression and secretion of r30 by rLm (see, e.g. PCT International Publication No. WO 2011/159814, the contents of which are incorporated by reference). We found that rLm30 significantly enhances BCG-primed protective efficacy against aerosol challenge with the virulent Mtb Erdman strain in mice and guinea pigs (15). Boosting BCG-primed C57BL/6 mice with rLm30 induces strong antigen-specific T-cell mediated immune responses, including greater frequencies of antigen-specific polyfunctional CD4+ and CD8+ T cells expressing interferon gamma (IFN-γ), tumor necrosis factor alpha (TNF-α), and/or Interleukin 2 (IL-2) in the spleens and lungs.

As discussed herein, we have determined that certain constellations of different M. tuberculosis proteins perform unexpectedly well when utilized in the immunogenic Listeria monocytogenes fusion protein compositions disclosed herein (i.e., can elicit significant protection against M. tuberculosis infection). Briefly, in efforts to expand the M. tuberculosis antigen repertoire of our rLm vaccine, we evaluated a number of different M. tuberculosis proteins in addition to r30 for potential inclusion in a multi-antigenic vaccine. We constructed a variety of new rLm multi-antigenic vaccine candidates including vaccines expressing a variety of immunogenic epitopes found on different M. tuberculosis proteins and evaluated them for efficacy against M. tuberculosis aerosol challenge in C57BL/6 and/or BALB/c mice. From these studies, we identified rLm5Ag (30), expressing the fusion protein 23.5 (MPT64)-TB10.4 (EsxH)-ESAT6 (EsxA)-CFP10 (EsxB)-r30 (Ag85B) as one of the most promising vaccine candidates. We then studied the immunogenicity of rLm5Ag (30) in BCG-primed C57BL/6 and BALB/c mice. We found that while the rLm-vectored multi-antigenic vaccine boosts BCG-primed protection in both C57BL/6 and BALB/c mice, this rLm vaccine induces strong T-cell mediated immune responses, evidenced by enhanced antigen-specific frequencies of splenic and lung CD4+ and CD8+ T cells expressing IFN-γ, TNF-α, and/or IL-2, in BCG-primed C57BL/6 mice but not in BALB/c mice, where such responses are markedly limited. Thus, while the rLm vaccines enhance protective immunity in both BCG-immunized mouse strains, they do so via disparate immune responses.

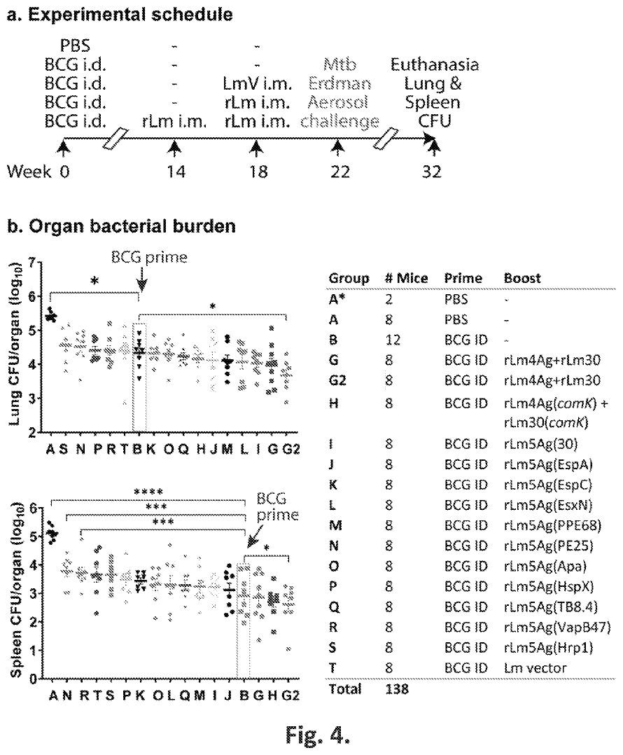

In the studies that led to the invention disclosed herein, we discovered that certain immunogenic epitopes present on selected M. tuberculosis proteins can significantly enhance the protective efficacy of vaccines against M. tuberculosis (e.g., in the Listeria monocytogenes fusion protein platforms disclosed herein). Significantly in these studies, the inventors discovered some M. tuberculosis protein immunogenic epitopes do not function well in the Listeria monocytogenes fusion protein vaccine compositions disclosed herein. For example, immunogenic epitopes found in certain M. tuberculosis proteins (e.g., Hrp1, PE25, HspX, and VapB47) actually compromised the ability of Listeria monocytogenes fusion protein vaccine compositions to prevent or inhibit M. tuberculosis infection (see, e.g, FIG. 4). In view of these discoveries, the fusion proteins disclosed herein comprise unique constellations of immunogenic epitopes selected for their unexpectedly effective ability to prevent or inhibit M. tuberculosis infection when used in vaccine compositions. Illustrating this, when administered as standalone vaccines, the Listeria vectored vaccines, expressing only 5 or 9 M. tuberculosis antigens (e.g. SEQ ID NO: 1 and 2) were able to induce protection against M. tuberculosis that is comparable to the protection obtained with the Bacille Calmette-Guérin (BCG) vaccine (which shares thousands of immunogenic epitopes with M. tuberculosis) (see e.g. Jia et al., Communications Biology 2022 Dec. 20; 5 (1): 1388; Lines 329-333, 355-367, 374-378 (mice); 436-438 (guinea pigs); Summary 519-522). This surprising observation illustrates the potential therapeutic efficacy of the specific vaccine compositions disclosed herein. The BCG vaccine has existed for over 80 years and is one of the most widely used of all current vaccines for limiting Mycobacterium tuberculosis infection. The BCG vaccine has a documented protective effect against meningitis and disseminated tuberculosis in children.

Embodiments of the invention include composition of matter comprising at least one fusion protein having antigenic epitopes present in at least two (and up to 9) Mycobacterium tuberculosis proteins selected from: 23.5/Mpt64, TB10.4/EsxH, ESAT6/EsxA, CFP10/EsxB, and r30/Antigen 85B; EsxN; PPE68; EspA and TB8.4. In certain embodiments of the invention, the composition comprises at least two fusion proteins having antigenic epitopes present in at least two (and up to 9) Mycobacterium tuberculosis proteins selected from: 23.5/Mpt64, TB10.4/EsxH, ESAT6/EsxA, CFP10/EsxB, and r30/Antigen 85B; EsxN; PPE68; EspA and TB8.4. In some embodiments of the invention, the at least one fusion protein does not comprise immunogenic epitopes present in at least one Mycobacterium tuberculosis protein selected from Hypoxic response protein 1 (“Hrp1”, UniProt P9WAJ3), PE-PGRS family protein PE25 (“PE25”, UniProt 16X486), Alpha-crystallin (“HSPX” UniProt P9WMK1) and Antitoxin VapB47 “VapB47” UniProt P9WF22). In certain embodiments of the invention, immunogenic epitopes are disposed on the fusion protein such that immunogenic epitopes of 23.5/Mpt64 are N-terminal to other Mycobacterium tuberculosis immunogenic epitopes disposed in the fusion protein. In certain embodiments of the invention, immunogenic epitopes are disposed on the fusion protein such that immunogenic epitopes of r30/Antigen 85B are C-terminal to other Mycobacterium tuberculosis immunogenic epitopes disposed in the fusion protein.

Typically, these compositions comprise Listeria monocytogenes expressing the at least one fusion protein or the at least two fusion proteins (e.g. a strain of Listeria monocytogenes that does not express a functional InlB protein; that does not express a functional actA protein; and/or expresses prfA protein having a G155S substitution mutation). In certain compositions of the invention, the Listeria monocytogenes expresses a first fusion protein encoded by a polynucleotide present in a first locus of the Listeria monocytogenes genome and a second fusion protein encoded by a polynucleotide present in a second locus of the Listeria monocytogenes genome. In one illustrative embodiment of the invention, the first fusion protein comprises epitopes present in at least two Mycobacterium tuberculosis proteins selected from: 23.5/Mpt64, TB10.4/EsxH, ESAT6/EsxA, CFP10/EsxB, and r30/Antigen 85B; and the second fusion protein comprises epitopes present in at least two Mycobacterium tuberculosis proteins selected from EsxN; PPE68; EspA and TB8.4.

In illustrative working embodiments of the invention, a fusion protein comprises antigenic epitopes present in the polypeptide sequence:

(SEQ ID NO: 1)
VGLNRFMRAMMVVFITANCITINPDIIFAATDSEDSSLNTDEWEEEKTE
EQPSEVNTGPRYETAREVSSRDIEELEKSNKVKNTNKADLIAMLKAKAE
KGGSMAPKTYCEELKGTDTGQACQIQMSDPAYNINISLPSYYPDQKSLE
NYIAQTRDKFLSAATSSTPREAPYELNITSATYQSAIPPRGTQAVVLKV
YQNAGGTHPTTTYKAFDWDQAYRKPITYDTLWQADTDPLPVVFPIVQGE
LSKQTGQQVSIAPNAGLDPVNYQNFAVTNDGVIFFFNPGELLPEAAGPT
QVLVPRSAIDSMLARPMSQIMYNYPAMLGHAGDMAGYAGTLQSLGAEIA
VEQAALQSAWQGDTGITYQAWQAQWNQAMEDLVRAYHAMSSTHEANTMA
MMARDTAEAAKWGGGGSGMTEQQWNFAGIEAAASAIQGNVTSIHSLLDE
GKQSLTKLAAAWGGSGSEAYQGVQQKWDATATELNNALQNLARTISEAG
QAMASTEGNVTGMFAGSSGGSSGMAEMKTDAATLAQEAGNFERISGDLK
TQIDQVESTAGSLQGQWRGAAGTAAQAAVVRFQEAANKQKQELDEISTN
IRQAGVQYSRADEEQQQALSSQMGFGSSGGSSGAFSRPGLPVEYLQVPS
PSMGRDIKVQFQSGGNNSPAVYLLDGLRAQDDYNGWDINTPAFEWYYQS
GLSIVMPVGGQSSFYSDWYSPACGKAGCQTYKWETFLTSELPQWLSANR
AVKPTGSAAIGLSMAGSSAMILAAYHPQQFIYAGSLSALLDPSQGMGPS
LIGLAMGDAGGYKAADMWGPSSDPAWERNDPTQQIPKLVANNTRLWVYC
GNGTPNELGGANIPAEFLENFVRSSNLKFQDAYNAAGGHNAVFNFPPNG
THSWEYWGAQLNAMKGDLQSSLGAG;

and/or
a fusion protein comprises antigenic epitopes present in the polypeptide sequence:

(SEQ ID NO: 2)
VGLNRFMRAMMVVFITANCITINPDIIFAATDSEDSSLNTDEWEEEKTE
EQPSEVNTGPRYETAREVSSRDIEELEKSNKVKNTNKADLIAMLKAKAE
KGGSMAPKTYCEELKGTDTGQACQIQMSDPAYNINISLPSYYPDQKSLE
NYIAQTRDKFLSAATSSTPREAPYELNITSATYQSAIPPRGTQAVVLKV
YQNAGGTHPTTTYKAFDWDQAYRKPITYDTLWQADTDPLPVVFPIVQGE
LSKQTGQQVSIAPNAGLDPVNYQNFAVTNDGVIFFFNPGELLPEAAGPT
QVLVPRSAIDSMLAMTINYQFGDVDAHGAMIRAQAASLEAEHQAIVRDV
LAAGDFWGGAGSVACQEFITQLGRNFQVIYEQANAHGQKVQAAGNNMAQ
TDSAVGSSWAGGSGMLWHAMPPELNTARLMAGAGPAPMLAAAAGWQTLS
AALDAQAVELTARLNSLGEAWTGGGSDKALAAATPMVVWLQTASTQAKT
RAMQATAQAAAYTQAMATTPSLPEIAANHITQAVLTATNFFGINTIPIA
LTEMDYFIRMWNQAALAMEVYQAETAVNTLFEKLEPMASILDPGASQST
TNPIFGMPSPGSSTPVGQLPPAATQTLGQLGEMSGPMQQLTQPLQQVTS
LFSQVGGTGGGNPADEEAAQMGLLGTSPLSNHPLAGGSGPSAGAGLLRA
ESLPGAGGSLTRTPLMSQLIEKPVAPSVMPAAAAGSSATGGAAPVGAGA
MGQGAQSGGSTRPGLVAPAPLAQEREEDDEDDWDEEDDWGSSGGSSGAM
SRAFIIDPTISAIDGLYDLLGIGIPNQGGILYSSLEYFEKALEELAAAF
PGDGWLGSAADKYAGKNRNHVNFFQELADLDRQLISLIHDQANAVQTTR
DILEGAKKGLEGEVWEFITNALNGLKELWDKLTGWVTGLFSRGWSNLES
FFAGVPGLTGATSGLSQVTGLFGAAGLSASSGLAHADSLASSASLPALA
GIGGGSGFGGLPSLAQVHAASTRQALRPRADGPVGAAAEQVGGQSQLVS
AQGSQGMGGPVGMGGMHPSSGASKGTTTKKYSEGAAAGTEDAERAPVEA
DAGGGQKVLVRNVVGSSGGSSGAMDPVDAVINTTCNYGQVVAALNATDP
GAAAQFNASPVAQSYLRNFLAAPPPQRAAMAAQLQAVPGAAQYIGLVES
VAGSCNNY.

Embodiments of the invention further include polynucleotides encoding the fusion proteins disclosed herein (e.g. SEQ ID NO: 1 or 2), for example such polynucleotides disposed within a vector

Embodiments of the invention also include methods of generating an immune response to a Mycobacterium tuberculosis comprising immunizing a mammal with a vaccine composition disclosed herein such that an immune response to Mycobacterium tuberculosis is generated. Those of skill in this art understand that the immunization methods disclosed herein can be combined with other methodological steps. For example, certain embodiments of the invention include the step of immunizing the mammal with Mycobacterium bovis strain Bacille Calmette-Guérin (BCG). Optionally in these embodiments, the BCG is used in a primary immunization and the Listeria monocytogenes expressing the at least one fusion protein or the at least two fusion proteins is used in a booster immunization. In such embodiments of the invention, the mammal can be immunized intradermally, intranasally, orally, subcutaneously, percutaneously, intramuscularly, intravenously, or by another conventional route of vaccine delivery. For example, in some of these methods the composition is administered subcutaneously. In certain methods, the composition is administered intradermally.

Further aspects and embodiments of the invention are discussed in the examples below.

### Example 1: Listeria-Vectored Multiantigenic Tuberculosis Vaccine Enhances Protective Immunity Against Aerosol Challenge with Virulent Mycobacterium Tuberculosis in BCG-Immunized C57BL/6 and BALB/C Mice

Aspects and embodiments of the invention discussed in this example are also discussed in Jia Q, Masleša-Galić S, Nava S, Horwitz M A. mBio. 2022 Jun. 28; 13 (3): e0068722. doi: 10.1128/mbio.00687-22. Epub 2022 Jun. 1, the contents of which are incorporated herein by reference.

### Material and Methods

### Cell lines, bacteria, and animals

Murine (J774A.1, ATCC TIB-67) and human (THP1, ATCC TIB-202) monocytes were differentiated into macrophage-like cells and cultured in Dulbecco's modified Eagle's medium (DMEM) and RMPI 1640 (RPMI) medium, respectively, containing penicillin (100 μg/ml) and streptomycin (100 U/ml) and supplemented with 10% fetal bovine serum (FBS). Mycobacterium bovis BCG Tice was purchased from Organon. M. tuberculosis (Mtb) Erdman strain (ATCC 35801) was harvested from infected outbred guinea pigs to verify virulence, cultured on 7H11 agar, subjected to gentle sonication to obtain a single cell suspension, and frozen at −80° C. for use in animal challenge experiments. All Listeria vector and recombinant Listeria-vectored vaccine stocks were grown to mid log phase in Yeast Extract broth medium and the bacteria collected by centrifugation, resuspended in phosphate buffer saline (PBS), titrated, and stored in 20% glycerol/PBS at −80° C. until use. Six to eight-week-old female C57BL/6 mice were purchased from Harlan (currently Envigo, Livermore, CA, USA) or Jackson Laboratory (Bar Harbor, Maine, USA) and BALB/c mice purchased from Jackson Laboratory.

### Construction and Verification of Lm-Vectored Multi-Antigenic Vaccines

We constructed Lm-vectored multi-antigenic rLm vaccine candidates using the Lm ΔactA ΔinlB prfA* vector, as we previously described (15). Briefly, to construct rLm vaccine candidates expressing Mtb multi-antigenic proteins, we analyzed the protein sequences of the selected 15 Mtb proteins, removed the predicted signal peptides of TB8.4 (2R-28A), Apa (2H-39A), r30/Ag85B (2Q-43A), and 23.5/Mpt64 (1V-23A) and the internal regions of HspX (1211-128V), PE25 (661-73L), and EspA (111F-193L) that might interfere with protein secretion from the rLm vaccine constructs; we kept the full-length sequences for TB10.4, EsxN, Hrp1/Rv2626c, VapB47/RV3407, EspC, PPE68, CFP-10, and ESAT-6. We optimized the coding sequence for each of the selected proteins for expression in Lm, purchased them from DNA2.0 (Newark, CA), and assembled the optimized DNAs encoding the indicated multi-antigenic proteins with or without a spacer encoding a GGSG (SEQ ID NO: 3) or GSSGGSSG (SEQ ID NO: 4) linker by traditional molecular cloning methods. We cloned the final assembled DNAs into a phage-based Listeria site-specific integration vector derived from pPL1 (kindly provided by P. Lauer) or pPL2e (kindly provided by J. Skoble) (40) downstream of the Lm actA promoter and ligated in-frame to the C-terminus of the ActAN. Subsequently, we integrated the Mtb antigen expression cassette together with the pPL vector into the comK locus or the 3′ the end of the tRNAarg locus on the bacterial chromosome of the recipient Lm vector, as described previously by us (15) and Lauer et al. (10, 16). All molecular plasmid constructs were confirmed by restriction enzyme digestion and nucleotide sequencing. The final rLm strains were verified by PCR using primers, specifically amplifying a unified 548-bp PCR product in strains that contain an integration vector at the bacterial attachment site tRNAarg-attBB′, and using primers, specifically amplifying a PCR product across the inserted gene with various sizes in different strains; the PCR products were further confirmed by nucleotide sequencing.

### Growth Kinetics of rLm Multi-Antigenic Vaccines in Broth Culture and in Murine and Human Macrophage-Like Cells

The growth kinetics of rLm vaccine candidates in broth culture and macrophage-like cells was examined as described by us previously with modifications (15). Glycerol stocks of the Lm vector and rLm vaccine candidates were inoculated into Brain Heart Infusion (BHI) medium supplemented with Streptomycin (200 μg/ml) (the Lm vector is streptomycin resistant) to prevent any contamination and grown overnight under stationary conditions in a 37° C. incubator with 5% CO2. The overnight culture was inoculated into 5 ml fresh BHI with Streptomycin at an initial optical density at 540 nm (OD540) of ˜0.05 and incubated at 37° C. with shaking at 180 rpm. At 0, 3, 5, and 7 hours post inoculation, a 1-ml aliquot of each culture was removed and measured for OD540.

The growth kinetics in macrophage-like cells was assayed by infecting monolayers of murine macrophage-like cells (J774A.1) or phorbol 12-myristate 13-acetate (PMA) differentiated monolayers of human macrophage-like cells (THP-1) with the Lm vector or rLm candidates cultured overnight to stationary phase at a multiplicity of infection of 1:10 for 90 min in DMEM (J774A.1) or RPMI (THP-1) medium supplemented with 10% heat-inactivated FBS (HI-FBS). After 90 min infection, cells were washed three times with PBS supplemented with 2% HI-FBS. The infected cells were cultured for an additional 4.5 hours in DMEM or RPMI supplemented with 10% HI-FBS and gentamycin (10 μg/ml). At 0, 2, 4, and 6 hours post infection, the medium was removed; the monolayers lysed with 0.1% Saponin/PBS; and the cell lysates serially diluted in PBS and plated on BHI agar plates supplemented with streptomycin (200 μg/ml). The plates were incubated at 37° C. for two days and colonies enumerated.

Immunization and Aerosol Challenge of Mice with Virulent M. tuberculosis Erdman Strain

Groups of BALB/c or C57BL/6 mice, 8 or 12/group, were primed with BCG intradermally (i.d.) or intranasally (i.n.). BCG-primed mice were either not boosted or boosted once or twice with 2×10⁶ CFU of a single rLm vaccine or combination of two rLm vaccine candidates expressing multiple Mtb antigens and challenged 3 or 4 weeks later by exposure to aerosolized Mtb Erdman strain generated by a Collison Type-6 Jet Nebulizer (CH Technologies USA, Waltham, MA) from 10 ml of Mtb bacterial suspension (1.6-2.6×10⁵ CFU/ml) for 30 min followed by 5 min to allow for settling of bacteria. The challenge dose was verified by euthanizing two animals and assaying CFUs in their entire lungs at Day 1 post challenge. The mice were euthanized at various times post-challenge, and the spleens and lungs removed and assayed for bacillus burden as described by us previously (15).

### Immunization of Mice and Assay for Intracellular Cytokine Staining of Mouse Spleen and Lung Cells

To determine the immunogenicity of rLm5Ag (30) expressing the fusion protein of 5 Mtb antigens (23.5-10.4-ESAT6-CFP10-r30) as a booster vaccine, we immunized C57BL/6 and BALB/c mice, 4/group, subcutaneously (s.q.) with BCG at Week 0; boosted them at Weeks 14 and 18 with 2×10⁶ CFU of the Lm vector or rLm5Ag (30); euthanized the mice at 6 days post the last immunization; prepared single cell suspensions of spleen and lung cells; stimulated the single cell suspensions with a single Mtb antigen or pool of multiple Mtb antigens; and assayed T-cell immunity by intracellular cytokine staining (ICS) using methods that we published previously (15, 17) with modifications as described below.

We conducted ICS by using an eight-color flow cytometry panel to simultaneously analyze multiple cytokines at the single-cell level. Specifically, a single cell suspension of 5×10⁵ lung cells per well or 1.0×10⁶ splenocytes per well was seeded in U-bottom 96-well plates and stimulated with medium alone (negative control), 5 μg/ml of recombinant proteins r30/Ag85B (our lab stock, isolated from recombinant Mycobacterium smegmatis), ESAT6/EsxA (BEI Resources), CFP10/EsxB (BEI Resources), TB10.4/EsxH (Aeras), 23.5/Mpt64 (BEI Resources), pool of 5 antigens (5Ag) comprising r30, ESAT6, CFP10, TB10.4, and 23.5, each at 2 μg/ml, or PPD (5 μg/ml) in the presence of anti-CD28 monoclonal antibody (Clone 37.51) for a total of 6 h. Cells stimulated with PMA served as a positive control. Four hours prior to harvest, GolgiPlug (protein transport inhibitor containing Brefeldin A) diluted in T-cell medium was added to all wells; PMA was additionally added to positive control wells. Following in vitro stimulation, cells were harvested, washed with PBS, incubated with Live/Dead Fix Near IR Cell Stain (Invitrogen) for 10 min at room temperature to identify dead cells, and surface stained with antibodies against CD4 (Clone RM4-5, conjugated with Brilliant Violet 510) and CD8 (Clone 53-6.7, conjugated with Brilliant Violet 605). Cells were then fixed/permeabilized with Cytofix/Cytoperm (BD BioSciences) and stained for CD3 (clone 17A2, conjugated with Alexa Fluor 488), IFN-γ (Clone XMG1.2, conjugated with Brilliant Violet 650), IL-2 (Clone JES6-5H4, conjugated with PE), TNF-α (Clone MP6-XT22, conjugated with PerCPCy5.5) and IL-17A (Clone TC11-18H10.1, conjugated with Alexa Fluor 647). Note that due to the internalization of CD3 in responding CD4+ T cells, cells were stained for CD3 after fixing/permeabilization. The Fluorochrome-conjugated antibodies were purchased from BioLegend. For Flow cytometry analysis, a minimum of 100,000 lymphocytes per sample was acquired with an LSRII-HT (BD) flow cytometer. The data were analyzed using FlowJo software. Initial gating of total events included a lymphocyte gate, followed by selection for singlet cells and live CD3+ T cells (Near IR-AF488+); CD4+ and CD8+ T cells were identified by CD4+ (BV510+BV605-) and CD8+ (BV605+BV510-) expression, respectively. The gates for frequencies of antigen-specific IFN-γ, IL-2, TNF-α, and IL-17A producing CD4+ and CD8+ T cells were determined by using the unstimulated cells; Boolean combinations of the four intracellular cytokine gates were used to uniquely discriminate responding cells based on their frequency with respect to cytokine production. Each cytokine-positive cell was assigned to one of the 15 possible combinations. Background frequencies of CD4+ and CD8+ T cells producing cytokines without antigen stimulation were subtracted. Two-way ANOVA with Sidak's multiple comparisons test was performed using GraphPad Prism 8.02 (San Diego, CA) to determine significance in comparisons of mean frequencies of cytokine-producing CD4+ and CD8+ T cells between mice vaccinated with the Lm vector and mice vaccinated with rLm5Ag (30).

### Statistical Analyses

One-way or two-way ANOVA with Tukey's multiple comparisons test was performed using GraphPad Prism 9.02 (San Diego, CA) to determine significance in comparisons of mean frequencies of cytokine producing CD4+ and CD8+ T cells and mean organ CFUs in spleens and lungs among mice in vaccinated and control groups.

### Results

Construction and verification of new rLm vaccines expressing 1, 3, or 4 recombinant Mtb proteins

Previously we have shown that rLm30, expressing the r30/Ag85B downstream of the Lm actA promoter and ligated to the N-terminal 100 amino acids of ActA (ActAN) as a fusion protein, boosts BCG-primed efficacy against TB (15). To expand the Mtb antigen repertoire of the rLm vaccine platform, we initially constructed 3 new rLm vaccine candidates-rLm23.5, expressing the mature peptide of 23.5/Mpt64A1V-23A); rLm3Ag, expressing the fusion protein of Ag85B (Δ2Q-43A)-RP-TB10.4-GGSG (SEQ ID NO: 3)-ESAT6 [RP, a dipeptide encoded by EagI restriction enzyme site for cloning purposes; GGSG (SEQ ID NO: 3), a flexible fusion protein linker]; and rLm4Ag, expressing the fusion protein of Mpt64 (A1V-23A)-RP-TB10.4-GGSG (SEQ ID NO: 3)-ESAT6-GSSGGSSG (SEQ ID NO: 4)-CFP10 (GSSGGSSG, (SEQ ID NO: 4) a flexible linker) (Table 1). The Mtb antigens in each vaccine construct were expressed as a C-terminal fusion protein to Lm ActAN; the Mtb protein expression cassette was driven by the Lm actA promoter and integrated at the tRNAarg of the Lm ΔactA ΔinlB prfA* chromosome.

TABLE 1
rLm vaccine candidates

| | Antigen expression | Estimated | Integration |
|---|---|---|---|
| Vaccine | cassette | pI^{a)}/Mw (Da) | locus |
| rLm23.5 | ActAN-Mpt64(Δ1V-23A) | 4.54/30560 | tRNA^{arg} |
| rLm3Ag | ActAN-Ag85B(Δ2Q-43A)- | 4.65/59691 | tRNA^{arg} |
| | TB10.4-ESAT6 | | |
| rLm4Ag | ActAN-Mpt64(Δ1V-23A)- | 4.57/62683 | tRNA^{arg} |
| | TB10.4-ESAT6-CFP10 | | |

^{a)}Isoelectric point

When referring to certain figures as “sFig” in the text in this Example, see the supplemental figures found in Jia Q, Masleša-Galić S, Nava S, Horwitz MA. mBio. 2022 Jun. 28; 13 (3): e0068722. doi: 10.1128/mbio.00687-22. Epub 2022 June, the contents of which are incorporated by reference. We verified the expression of the heterologous protein ActAN-Mpt64 by rLm23.5 as a 31-kDa protein band detected by a polyclonal antibody to a peptide comprising 18 amino acids (A30-K47) of ActAN (AK18) (courtesy of J. Skoble and P. Lauer) (sFIG. 1a); expression of ActAN-Ag85B-TB10.4-ESAT6 by rLm3Ag as a 59-kDa protein band detected by a rabbit polyclonal antibody to r30 (sFIG. 1b, upper panel) or a polyclonal antibody to TB10.4 (sFIG. 1b, lower panel), and expression of ActAN-Mpt64-TB10.4-ESAT6-CFP10 by rLm4Ag as a 63-kDa protein band detected by AK18 (sFIG. 1c).

Boosting BCG-primed C57BL/6 mice with the combined rLm30-10.4-ESAT6 and rLm23.5 vaccines enhances protection against aerosolized Mtb.

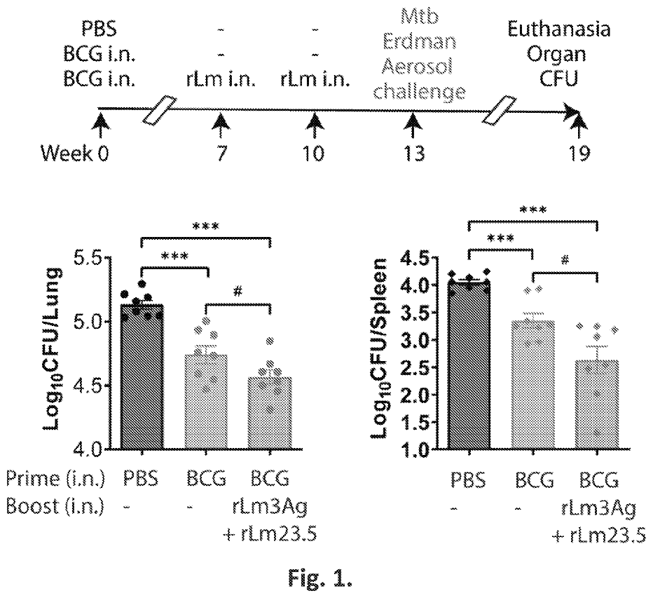

To determine the efficacy of these multi-antigenic vaccine candidates as a booster vaccine in protecting BCG-immunized mice against aerosolized Mtb challenge, we immunized C57BL/6 mice, 8/group, i.n. with PBS (sham) or BCG at Week 0 and boosted one group of BCG-immunized mice i.n. with rLm30-10.4-ESAT6 (rLm3Ag)+rLm23.5 at Weeks 7 and 10. The mice were then challenged with aerosolized Mtb Erdman (2.6×10⁵ CFU for 30 min resulting in an average of 21 CFU in the lungs at Day 1 post challenge) at Week 13, euthanized at Week 19, and their lungs and spleens assayed for Mtb CFU. As shown in FIG. 1, mice primed-boosted with BCG-rLm3Ag+rLm23.5 had a significantly lower bacterial burden in their lungs and spleens than sham-immunized mice and mice immunized with BCG. This result indicates that boosting BCG-primed C57BL/6 mice with a rLm multi-antigenic vaccine enhances immunoprotection against Mtb aerosol challenge.

Boosting BCG-primed BALB/c mice with the combined rLm23.5-10.4-ESAT6-CFP10 and rLm30 vaccines enhances protection against aerosolized Mtb.

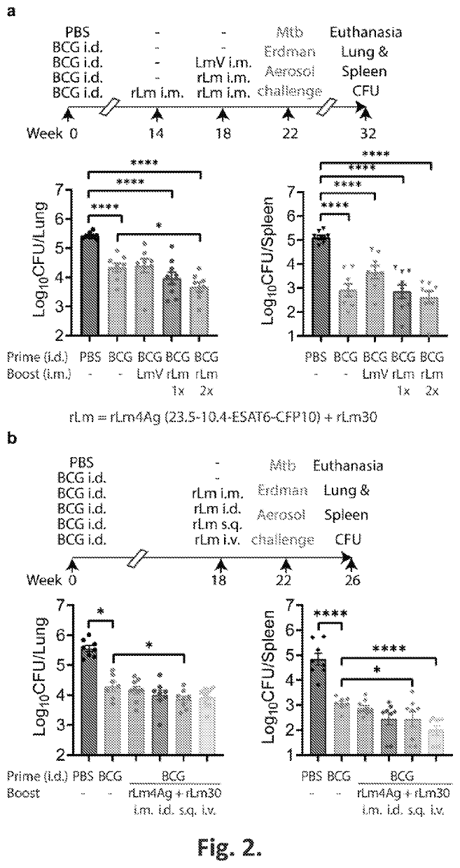

To further verify the immunoprotection against Mtb challenge of multi-antigenic rLm vaccine candidates as a booster vaccine in BCG-immunized mice, we immunized and challenged a different strain of mice, BALB/c mice. We immunized BALB/c mice, 8/group, i.d. with PBS (sham) or BCG at Week 0, did not boost or boosted BCG-immunized mice intramuscularly (i.m.) with Lm vector (LmV) or rLm23.5-TB10.4-ESAT6-CFP10 (rLm4Ag)+rLm30 at Weeks 14 and 18. The mice were then challenged with aerosolized Mtb Erdman (1.6×10⁵ CFU for 30 min resulting in an average of 19 CFU in the lungs at Day 1 post challenge) at Week 22, euthanized at Week 32, and their lungs and spleens assayed for Mtb CFU (FIG. 2a, upper panel). As shown in FIG. 2a, bottom panels, mice primed i.d.-boosted i.m. with BCG-rLm multi-antigenic (5 antigens in total) vaccine had significantly lower CFUs in their lungs (P<0.0001) and spleens (P<0.0001) than the sham-immunized mice. Of note, mice primed i.d.-boosted i.m. twice with BCG-rLm vaccine had significantly lower CFU in their lungs than mice primed i.d. with BCG alone (P<0.05). These results verify in BALB/c mice that boosting BCG-primed mice with an rLm multi-antigenic vaccine enhances immunoprotection against Mtb aerosol challenge.

In a companion experiment challenged at the same time as the experiment described in FIG. 2a, we compared different delivery routes for the rLm multi-antigenic booster vaccines. We immunized BALB/c mice, 8/group, i.d. with 5×10⁵ CFU of BCG at Week 0, and boosted them once at Week 18 with rLm23.5-10.4-ESAT6-CFP10 (rLm4Ag)+rLm30 once via the i.d., i.v., subcutaneous (s.q.), or intramuscular (i.m.) route. Mice immunized i.d. with PBS (Sham) or BCG at Week 0 and not boosted served as controls. At Week 22, we challenged the mice with aerosolized Mtb Erdman (as described in the companion experiment above). At Week 26, we euthanized the mice and assayed bacillus burdens in their lungs and spleens (FIG. 2b, upper panel).

As shown in FIG. 2b, bottom panels, priming with BCG i.d. alone was highly effective in comparison with Sham immunization, reducing CFU by 1.3 log in the lung and 1.8 log in the spleen. Despite the especially high efficacy of BCG alone, BCG prime-rLm boosting further reduced CFUs in the lungs and spleens after aerosol challenge with Mtb; the differences in CFU between boosted and non-boosted mice were statistically significant in the lungs and spleens for boosting via the s.q. route (P<0.05) and in the spleens for boosting via the s.q. (P<0.05) and i.v. routes (P<0.0001). Among the 4 boosting routes tested, the effectiveness in reducing lung CFU by route was s.q.>i.v.>i.d.>i.m., lowering CFU by 0.43, 0.37, 0.29, and 0.13 logs, respectively, vs. BCG alone, and the effectiveness in reducing spleen CFU by route was i.v.>s.q.>i.d.>i.m., lowering CFU by 1.07, 0.63, 0.61, and 0.18 logs, respectively, vs. BCG alone. Thus, the most effective routes were s.q. and i.v., and s.q. was the superior route in the lung. Surprisingly, the i.m. route was the least efficacious in both the lung and spleen. However, in the companion experiment described above (FIG. 2a), boosting i.m. twice with rLm4Ag+rLm30 provided improved protection in the lung (P<0.05) and spleen compared with boosting once. Given the safety and practical advantage of the s.q. route of administration and the fact that the s.q. route was superior to other routes in the lung, the major site of Mtb pathology, we selected the s.q. route for future studies.

TABLE 2
Fifteen Mtb proteins selected as vaccine candidates

| | | | Protection | | |
|---|---|---|---|---|---|
| | | length | in animal | Absence | Homologs |
| Rv # | Product (18) | (aa) | models^{a} | in BCG | in BCG^{b, c} |
| 0288 | TB10.4/EsxH/CFP-7, ESAT6 family | 96 | (19) | No | 0328 |
| 1174c | TB8.4, low molecular-weight T-cell antigen | 110 | (20) | No | 1237c |
| 1793 | EsxN, Putative ESAT-6 like protein | 94 | | No | 1825 |
| 1860 | Apa, alanine and proline rich secreted glycoprotein | 325 | (21) | No | 1896 |
| 1886c | r30/Ag85B/FbpB, 30 kDa major secreted protein | 325 | (3, 22, 23) | No | 1923c |
| 1980c | 23.5/Antigen Mpt64 | 228 | (24, 25) | Yes | — |
| 2031 | HspX, heat shock protein HspX | 144 | (26, 27) | No | 2050c |
| | (a-crystallin homolog) | | | | |
| 2431c | PE25, Esx-5, secreted with PPE41 & EspG5 | 99 | | No | 2450c |
| 2626c | HrpI, hypoxic response protein 1 | 143 | (28) | No | 2653c |
| 3407 | Antitoxin VapB47, part of the toxin-antitoxin | 99 | (28, 29) | No | 3477 |
| | operon with Rv3408 | | | | |
| 3615c | EspC, ESX-1 secretion associated protein C | 103 | (30) | No | 3679c |
| 3616c | EspA, ESX-1 secretion associated protein A | 392 | (30) | No | 3680c |
| 3873 | PPE68, interacts with ESAT6, CFP10 & TB10.4 | 368 | (30) | Yes | — |
| 3874 | CFP-10/EsxB, co-transcribed with Rv3875 | 100 | (31) | Yes | — |
| 3875 | ESAT-6/EsxA, early secretory antigen target | 95 | (30, 32) | Yes | — |

^{a}As an individual protein
^{b}Blasted using nucleotide blast tool via https://blast.ncbi.nlm.nih.gov/Blast.cgi
^{c}M. bovis BCG reference strain: Pasteur 1173P2, gene bank access number AM408590.1

Selection of Mtb antigens and construction of 13 new rLm5Ag vaccine candidates

To further expand the Mtb antigen repertoire, we selected 15 Mtb proteins (including r30) as potential vaccine candidates for further investigation (Table 2), including a) Secreted proteins: r30 (3); 23.5/Mpt64/Rv1980c (24, 25, 30); TB8.4/Rv1174c (20); Apa/MPT32/Rv1860 (21); b) ESAT6 and associated proteins secreted by the Esx/Type VII secretion system: ESAT6/EsxA/Rv3875 (30, 32), CFP10/EsxB/Rv3874 (31), TB10.4/EsxH/Rv0288 (19, 33), EspA/Rv3616c (30), EspC/Rv3615c (30), and EsxN/Rv1793; c) Antigenic PE/PPE proteins: PE25/Rv2431c (34) and PPE68/Rv3873 (30, 35); and d) Latency associated proteins: α-crystallin/hspX/Rv2031c (26, 27), Hrpl/Rv2626c (28), and VapB47/Rv3407 (28, 29). Among the 15 selected proteins, all but two (EsxN and PE25) have been shown by us or others to be immunoprotective antigens when incorporated into various vaccines including protein/adjuvant, DNA, Listeria-vectored or virus-vectored vaccines and 4 proteins-23.5/Mpt64, PPE68, CFP-10, and ESAT-6-are absent either from all BCG strains or the modern BCG strain (Mpt64) (36, 37) (Table 2).

We constructed 11 new rLm5Ag vaccine candidates carrying a single copy of an ActAN-Mtb 5 antigen fusion protein expression cassette downstream of the Lm actA promoter integrated at the 3′ end of the tRNAarg locus in the rLm chromosome; in all such cases, the first four proteins in the fusion protein were Mpt64-EsxH-EsxA-EsxB followed by a GSSGGSSG (SEQ ID NO: 4) flexible linker and the fifth protein was one of the 11 other selected Mtb proteins in Table 2. In addition, we constructed 2 rLm vaccine candidates expressing the Mtb 4Ag fusion protein or r30/Ag85B from the comK locus of the rLm chromosome, rLm4Ag (comK) and rLm30 (comK), respectively, to allow a comparison of vaccines expressing proteins at this locus vs. the tRNAarg locus and to explore the possibility of later expressing proteins from both loci in the same vaccine (Table 3).

TABLE 3
13 new rLm multi-antigenic vaccine candidates

| | | Estimated | |
|---|---|---|---|
| | | pI/Mw | Integration |
| Vaccine | Mtb antigen expression cassette ^{a)} | (Da) ^{b)} | locus |
| rLm5Ag(30) | ActAN-Mpt64-EsxH-EsxA-EsxB- | 4.63/94106 | tRNA^{arg} |
| | r30(Δ2Q-43A) | | |
| rLm5Ag(EspA) | ActAN-Mpt64-EsxH-EsxA-EsxB- | 4.69/94889 | tRNA^{arg} |
| | EspA(Δ111F-193L) | | |
| rLm5Ag(EspC) | ActAN-Mpt64-EsxH-EsxA-EsxB-EspC | 4.64/74239 | tRNA^{arg} |
| rLm5Ag(EsxN) | ActAN-Mpt64-EsxH-EsxA-EsxB-EsxN | 4.59/73386 | tRNA^{arg} |
| rLM5Ag(PPE68) | ActAN-Mpt64-EsxH-EsxA-EsxB-PPE68 | 4.46/100774 | tRNA^{arg} |
| rLm5Ag(PE25) | ActAN-Mpt64-EsxH-EsxA-EsxB- | 4.67/73422 | tRNA^{arg} |
| | PE25(Δ66I-73L) | | |
| rLm5Ag(Apa) | ActAN-Mpt64-EsxH-EsxA-EsxB- | 4.55/92355 | tRNA^{arg} |
| | APA(Δ2H-39A) | | |
| rLm5Ag(HspX) | ActAN-Mpt64-EsxH-EsxA-EsxB- | 4.66/78888 | tRNA^{arg} |
| | HspX(Δ121I-126V) | | |
| rLm5Ag(TB8.4) | ActAN-Mpt64-EsxH-EsxA-EsxB- | 4.55/71912 | tRNA^{arg} |
| | TB8.4(Δ2R-28A) | | |
| rLm5Ag(VapB47) | ActAN-Mpt64-EsxH-EsxA-EsxB-VapB47 | 4.80/74453 | tRNA^{arg} |
| rLm5Ag(Hrp1) | ActAN-Mpt64-EsxH-EsxA-EsxB-Hrp1 | 4.65/78962 | tRNA^{arg} |
| rLm4Ag(comK) | ActAN-Mpt64-EsxH-EsxA-EsxB | 4.57/62683 | comK |
| rLm30(comK) | ActAN-Ag85B(Δ2Q-43A) | 4.67/38789 | comK |

^{a)} Mpt64 refers to Mpt64(Δ1V-23A)
^{b)} The estimated Mw of each fusion protein is calculated as a secreted form without the signal peptide for ActA (1V-29A, 3.3 kDa).

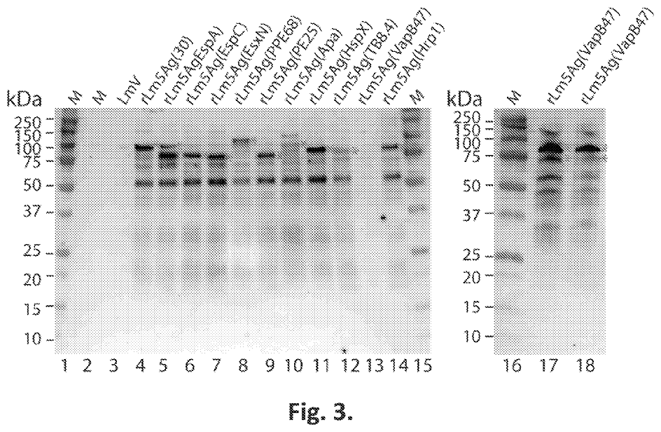

We examined Mtb fusion protein expression by rLm5Ag vaccine candidates grown in broth medium. As shown in FIG. 3, one major protein band (indicated by red asterisks to the right of the protein band) at the estimated Mw (Table 3, the 3rd column from the left) of the fusion protein and multiple minor protein bands (possible N-terminal protein breakdown products) were detected in the lysates of 10 rLm vaccine candidates (Lanes 4-12, and 14) by the rabbit Ak18 polyclonal antibody to ActAN. No specific protein band was detected from the lysate of the Lm vector (LmV, lane 3) as expected. One rLm vaccine candidate (lane 13), rLm5Ag (VapB47), also showed no protein expression in the lysate; however, two other clones of the same construct did express a protein of the approximately expected Mw (lanes 17 and 18). Similar protein bands were detected by the ActA AK18 antibody in the lysates of J774A.1 cells infected with the relevant rLm vaccine candidates. We also detected the Mtb protein expression by rLm4Ag (comK) and rLm30 (comK) grown in broth medium. Thus, we have verified that 13 new rLm vaccine candidates express a fusion protein of the expected size when grown in broth and macrophages.

We also verified the Mtb protein expression cassette integrated at the tRNAarg locus by PCR and nucleotide sequencing of the resultant PCR products. As shown in sFIG. 2a, amplification across the bacterial attachment site tRNAarg-attBB′ with primers NC16 and PL95 resulted in a 548-bp fragment in each selected clone of the rLm candidates (lanes 3-10, 12, 14); with regard to rLm5Ag (VapB47), 2 out of 3 clones selected tested positive (lanes 13, 15, and 16). Amplification with primers 319 and 327 across the antigen expression cassette resulted in various sizes of the PCR product, as shown in sFIG. 2b. Consistent with the PCR result using primers NC16 and PL95, a ˜2154 bp DNA fragment was amplified with primers 319 and 327 from 2 out of 3 selected clones of rLm5Ag (VapB47) (sFIG. 2b, lanes 13, 17 and 18).

We tested the genetic stability of the Mtb 5Ag expression cassette integrated into the rLm chromosome by culturing the vaccine candidates in the presence and absence of erythromycin, a marker used for selection of the rLm constructs. We observed that the Mtb 5Ag antigen expression cassettes were stable after passage in vitro in the absence of antibiotic selection, except for one clone of the rLm5Ag (VapB47), expressing Rv3407 as the fifth protein (sFIG. 3). Rv3407 encodes an antitoxin virulence associated protein B47 that is part of the toxin-antitoxin operon with Rv3408. Expressing Rv3407 independently of Rv3408 may have resulted in instability of this rLm fusion protein. However, when we grew the vaccine candidates carrying the antigen expression cassette including Rv3407 in the presence of antibiotic selection, these vaccines expressed the Mtb fusion proteins abundantly with two major bands of ˜74 (secreted form) and ˜78 kDa (non-secreted form) (FIG. 3, lanes 17 and 18).

Growth Kinetics of rLm5Ag Vaccine Candidates in Broth Medium and in Infected Murine and Human Macrophages

We examined the growth kinetics in BHI broth of the 11 new rLm5Ag vaccine candidates. As shown in sFIG. 4a-c, all of the rLm5Ag vaccine candidates, except rLm5Ag (VapB47) (this clone was subsequently discarded and replaced by a new clone shown in FIG. 3, lane 17), grew similarly in broth to the Lm vector. rLm4Ag (comK) and rLm30 (comK) also grew similarly to the Lm vector in broth medium.

To examine the growth kinetics of rLm5Ag vaccine candidates in macrophage-like cells, we infected monolayers of murine J774A.1 cells or monolayers of human THP-1 cells differentiated by PMA with the Lm vector or with the rLm vaccine candidates at a MOI of 10. In general, rLm5Ag vaccine candidates expressing fusion proteins comprising Mtb 4Ag (23.5-10.4-ESAT6-CFP10) ligated with a 5th antigen grew similarly to the Lm vector in both murine (sFIG. 4d-f) and human (sFIG. 4g-i) macrophage-like cells.

Protective Immunity of rLm5Ag Vaccine Candidates Against Aerosol Challenge with Virulent Mtb Erdman Strain in BALB/c Mice.

To screen for optimal Mtb antigens, we examined the protective efficacy against aerosolized Mtb of priming mice i.d. with BCG and boosting them i.m. (this experiment was initiated prior to our obtaining results of the experiment described above that determined that the optimal route was s.q.) with the 11 new rLm5Ag vaccine candidates each expressing the Mtb 4Ag fusion protein (ActAN-Mpt64-10.4-ESAT6-CFP10) ligated at its C-terminus with a new 5th antigen. We immunized BALB/c mice, 8 per group, i.d. with PBS (Sham) or i.d. with 5×10⁵ CFU of BCG at Week 0 and boosted them i.m. once at Week 18 with 2×10⁶ CFU each of the Lm Vector, 11 rLm5Ag candidates, or with one of two rLm vaccine combinations expressing the same 5 Mtb antigens as rLm5Ag (30). At Week 22, we challenged the mice with aerosolized Mtb (average of 19 CFU delivered to the lungs of each animal). At Week 32 (10 weeks post challenge), we euthanized the mice and assayed bacillus burdens in their lungs and spleens (FIG. 4a). Among the groups tested (FIG. 4b, right panel), Group G (rLm4Ag+rLm30) served as a control for Group I [rLm5Ag (30)] for comparison of the combination of two rLm vaccines (rLm4Ag+rLm30) with rLm5Ag (30) expressing Mtb 4Ag fused with r30; Group G2 served as a control for Group G for comparison of 1 (Week 18) vs. 2 (Week 14 and Week 18) boosts with this same combination of rLm vaccines; Group H served as a control for Group G for comparison of Mtb antigens expressed from comK locus [rLm4Ag (comK)+rLm30 (comK)] vs. tRNAarg locus [rLm4Ag+rLm30] in the Lm chromosome. As shown in FIG. 4b, a) the Lm vector (Group T) does not boost protective immunity induced by BCG alone; b) among the 13 rLm vaccines screened, the rLm vaccine candidates expressing 4Ag+r30 in various forms, i.e. rLm4Ag+rLm30, rLm4Ag (comK)+rLm30 (comK), and rLm5Ag (30) in Groups G, G2, H, and I, are the best booster vaccines; the lung CFUs in the mice boosted with these vaccines are lower than those in mice primed i.d. with BCG only, although the difference did not reach statistical significance for a single booster immunization [however boosting twice with rLm4Ag+rLm30 induced immunoprotection significantly greater than BCG alone in the lung (P<0.05)]; c) immunity induced by Mtb 5Ag expressed from a single rLm vaccine [rLm5Ag (30); Group I] is comparable to that induced by the same Mtb 5Ags expressed by two rLm vaccines administered together (rLm4Ag+rLm30; Group G), as evidenced by the equivalent CFUs in the lungs of mice in these two groups; d) immunity induced by the combination of two vaccines (rLm4Ag+rLm30) expressing Mtb 5Ag from the comK locus (Group H) is comparable to that induced by the combination of two parallel vaccines expressing Mtb 5Ag from the tRNAarg locus (Group G); and e) as noted, two boosts with rLm5Ag (rLm4Ag+rLm30) (Group G2) is more efficacious than one boost (Group G), as was similarly demonstrated in the experiment shown in FIG. 2a.

Overall, of the single vaccines expressing 5 Mtb antigens, we considered rLm5Ag (30) as the most efficacious, as it had the lowest CFU count in the lung, the major site of TB pathology, and the second lowest CFU count in the spleen. Hence, this vaccine was evaluated further for immunogenicity.

Boosting BCG-Primed Mice with rLm5Ag (30) Induces Disparate Antigen-Specific CD4+ and CD8+Immune Responses in C57BL/6 and BALB/c Mice.

To determine the immunogenicity of rLm5Ag (30) as a booster vaccine in BCG-primed mice, we primed C57BL/6 and BALB/c mice, 4/group, i.d. with 5×10⁵ CFU of BCG at Week 0 and boosted them s.q. twice at Weeks 14 and 18 with Lm Vector or rLm5Ag (30). At 6 days post the last immunization, we euthanized the mice, prepared single cell suspensions of spleen and lung cells, seeded the cells in 96-well cell-culture plates, stimulated the cells with various Mtb antigens, and assayed T-cell immunity by ICS.

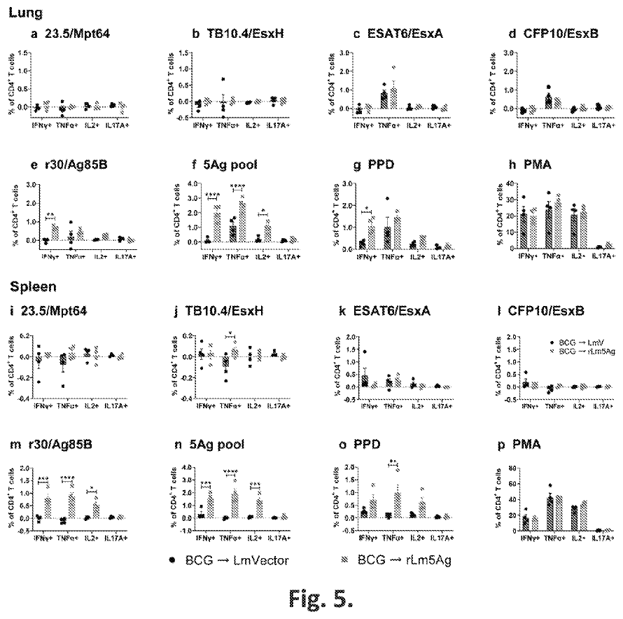

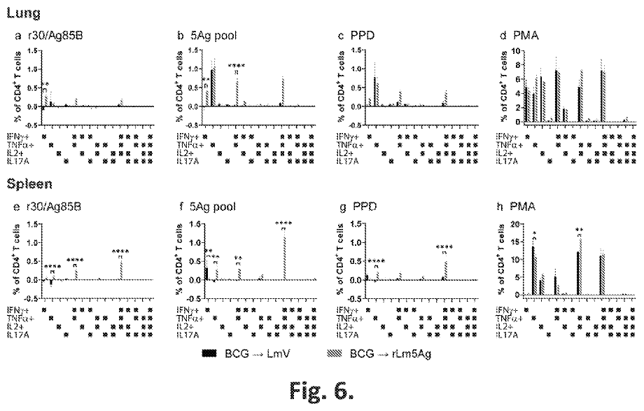

C57BL/6 mice, but not BALB/c mice, primed-boosted with BCG-rLm5Ag (30) produced a lower frequency of CD4+ T cells, greater frequency of CD8+ T cells, and lower CD4+/CD8+ T cell ratio than mice primed-boosted with BCG-LmVector in their lungs after in vitro stimulation without (Medium control) or with Mtb antigens (sFIG. 5). There are no significant differences in the frequencies of CD4+ and CD8+ T cells in the spleens of C57BL/6 and BALB/c mice (sFIG. 6). With respect to CD4+ T cells in the spleens and lungs of C57BL/6 mice, as shown in FIG. 5 and FIG. 6, mice primed with BCG and boosted with rLm5Ag (30) produce significantly greater frequencies of CD4+ T cells expressing intracellular cytokines IFN-γ, TNF-α, and/or IL-2 (FIG. 5) and polyfunctional CD4+ T cells expressing IFN-γ and TNF-α, or IFN-γ, TNF-α, and IL-2 (FIG. 6) in response to in vitro stimulation with r30/Ag85B, 5Ag pool, PPD, or TB10.4/EsxH than mice immunized with the Lm vector. No significant differences in the frequencies of CD4+ T cells expressing any of the cytokines were detected after in vitro stimulation with ESAT6/EsxA, CFP10/EsxB, and 23.5/Mpt64. As expected, mice immunized with the Lm vector and rLm5Ag (30) produced mostly comparable amounts of cytokines after in vitro stimulation with PMA (FIG. 5 & FIG. 6).

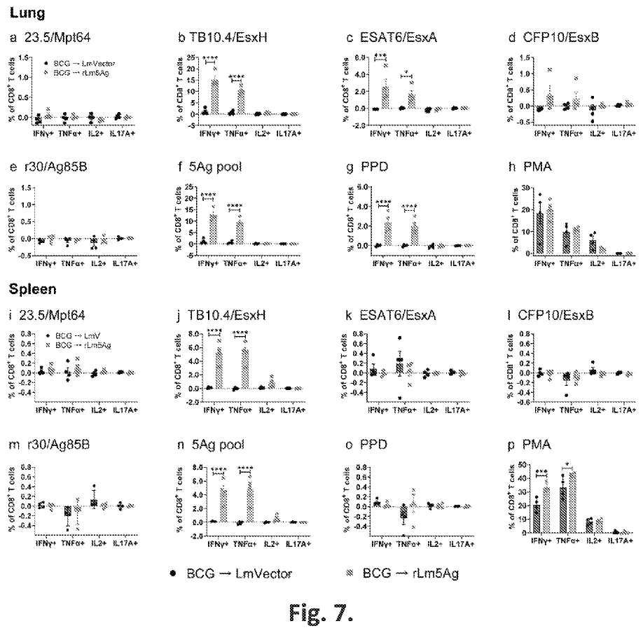

With respect to CD8+ T cells in spleens and lungs of C57BL/6 mice (FIG. 7), mice primed-boosted with BCG-rLm5Ag (30) produced significantly greater frequencies of CD8+ T cells expressing IFN-γ and TNF-α in response to in vitro stimulation with the TB10.4/EsxH (FIG. 7b, 7j) and the 5Ag pool (FIG. 7g, 70) in their spleens and lungs than mice immunized with the Lm vector. Two notable differences between spleens and lungs were that mice primed-boosted with BCG-rLm5Ag (30) produced significantly greater frequencies of CD8+ T cells expressing IFN-γ and TNF-α in response to in vitro stimulation with the ESAT6/EsxA (FIG. 7k) and PPD (FIG. 7n) in their lungs but not in their spleens (FIG. 7c, 7f); mice primed-boosted with BCG-rLm5Ag (30) produced significantly greater frequencies of CD8+ T cells expressing IFN-γ and TNF-α in their spleens (FIG. 7h) but not in their lungs (FIG. 7p) in response to in vitro stimulation with PMA than mice immunized with the Lm vector, but the relative differences in frequency and in statistical significance in the spleen were much less than for 5Ag and TB10.4/EsxH. No significant differences in the frequencies of spleen and lung CD8+ T cells expressing any of the cytokines were detected after in vitro stimulation with 23.5/Mpt64 (FIG. 7a, 7i), r30/Ag85B (FIG. 7e, 7m), or CFP10/EsxB (FIG. 7d, 71). These results show that boosting BCG-primed C57BL/6 mice with rLm5Ag (30) induces Mtb antigen specific CD4+ and CD8+ T cell-mediated immune responses.

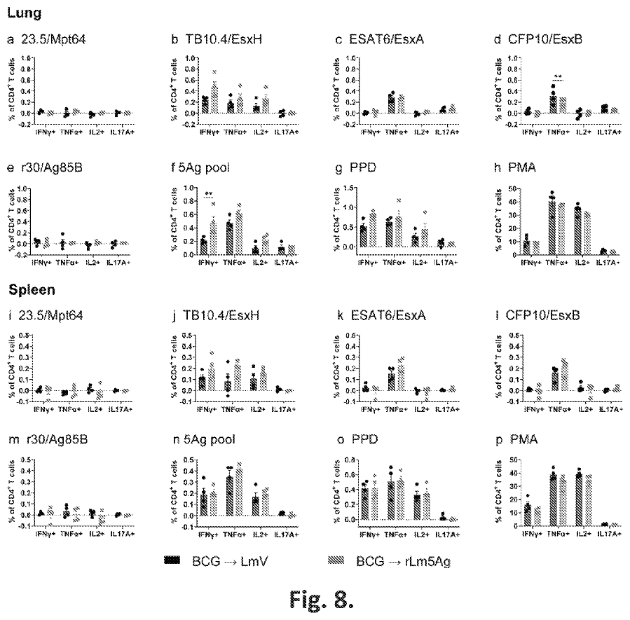

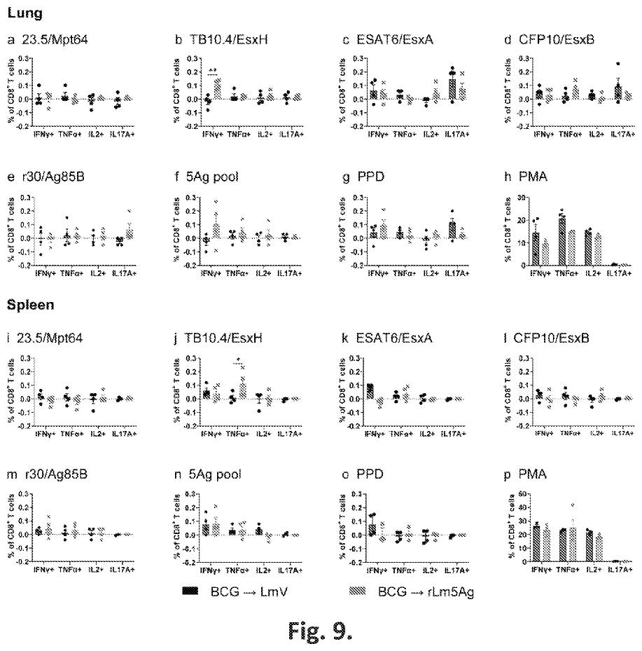

In a similar experiment performed in BALB/c mice (FIG. 8), in contrast to C57BL/6 mice, there were no significant differences between mice primed-boosted with BCG-LmV and BCG-rLm5Ag (30) in frequencies of splenic and lung CD4+ and CD8+ T cells expressing IFN-γ, TNF-α, IL2, or IL17 in response to in vitro stimulation with 23.5, TB10.4, ESAT6, CFP10, r30/Ag85B, PPD, and PMA; the only exception is that mice primed-boosted with BCG-rLm5Ag (30) had significantly greater frequencies of CD4+ T cells expressing IFN-γ in response to 5Ag pool stimulation in the lung (FIG. 8f). With respect to CD8+ T cells (FIG. 9), compared with mice primed-boosted with BCG-LmV, BALB/c mice primed-boosted with BCG-rLm5Ag (30) produced significantly greater frequencies of CD8+ T cells expressing IFN-γ or TNF-α in response to TB10.4 in their lungs (P<0.01) (FIG. 9b) and spleens (P<0.05) (FIG. 9j), but not to other Mtb antigens expressed by the rLm5Ag (30) vaccine (FIG. 9). These results indicate the rLm5Ag (30) multi-antigenic vaccine candidate induces a qualitatively and quantitatively different immune response in BCG-primed C57BL/6 and BALB/c mice.

### DISCUSSION

Our study shows that boosting BCG primed C57BL/6 and BALB/c mice with a Lm-vectored multi-antigenic Mtb vaccine candidate expressing combinations of M. tuberculosis proteins, especially rLm5Ag (30), expressing a fusion protein of r30/Ag85B, TB10.4/EsxH, ESAT6/EsxA, CFP10/EsxB, and 23.5/Mpt64, enhances the immunoprotection conferred by BCG against aerosol challenge with virulent M. tuberculosis Erdman strain in both mouse strains. Boosting C57BL/6 mice with rLm5Ag (30) significantly enhances the level of CD8+ T cell expression in the spleens and lungs, the frequency of multifunctional splenic CD4+ T cells expressing IFN-γ, TNF-α, and IL-2 in response to r30/Ag85B, PPD, and the 5Ag pool, and the frequency of splenic and lung CD8+ T cells expressing IFN-γ and TNF-α in response to TB10.4/EsxH and/or ESAT6/EsxA antigens. Although boosting BCG-primed BALB/c mice with rLm5Ag (30) also enhances the frequency of some cytokine-secreting lymphocytes, specifically splenic and lung CD8+ T cells expressing IFN-γ or TNF-α in response to TB10.4/EsxH antigen, the response is much more limited than in BCG-primed C57BL/6 mice.

Of the five recombinant Mtb antigens expressed by rLm5Ag (30), all have previously been demonstrated to be immunoprotective individually as well as in combination with other Mtb antigens. r30/Ag85B has been demonstrated to be highly protective when administered as an adjuvanted recombinant protein (22) or when expressed by recombinant BCG (rBCG30) (3, 23) or an Lm vector (15) in guinea pigs and mice. TB10.4 alone or as part of an Ag85B-TB10.4 fusion protein in adjuvant has been shown to induce protection in mice (19) and guinea pigs (38, 39). ESAT6, alone or in combination with Antigen 85B, administered with the adjuvant monophosphoryl lipid A has been shown to induce protective immunity in mice (32, 33). CFP10 delivered via a DNA vaccine induces protection against aerosolized Mtb Erdman in C3H/HeJ mice (31), and a Salmonella vectored vaccine expressing an ESAT6-CFP10 fusion protein protects C57BL/6 mice against aerosolized Mtb H37Rv (40). Finally, the 23.5/Mpt64 protein expressed by a DNA vaccine (24) or surface expressed by recombinant BCG (25) has been found to induce protective immunity in C57BL/6 mice challenged intravenously with H37Rv (24) or by aerosol with Mtb Erdman (25).

Notably, of the five antigens in rLm5Ag (30), three are absent from BCG entirely (ESAT6/EsxA and CFP10/EsxB) or from modern strains of BCG (23.5/Mpt64). Hence, boosting BCG with rLm5Ag (30) not only enhances the level of immunity to immunoprotective proteins present in BCG, but additionally broadens the immune response to encompass antigens present in M. tuberculosis but absent from BCG.

Also of note, the five proteins comprising rLm5Ag (30) are all secreted or extracellularly released proteins. Such extracellular proteins have been demonstrated to be especially important immunoprotective antigens of intracellular pathogens and hypothesized early on to play a central role in vaccines against such pathogens including Legionella pneumophila and Mycobacterium tuberculosis (33, 41, 42).

Our screen of 11 Listeria vectored vaccines expressing 5 Mtb antigens, all comprising a fusion protein of 23.5-TB10.4-Esat6-CFP10 plus one of 11 additional antigens, revealed several vaccine candidates that induced protection better than BCG and almost comparable to rLm5Ag (30). The most potent alternative “fifth” antigens were EsxN, PPE68, EspA and Tb 8.4. Of these, one antigen, PPE68, is absent from BCG. We have subsequently constructed a 9-antigen Listeria vectored vaccine incorporating these additional four Mtb antigens, and in on-going studies, we are evaluating it for protective efficacy in mice, guinea pigs, and non-human primates.

The rLm5Ag (30) vaccine induced significantly enhanced levels of antigen-specific cytokine-secreting CD4+ and CD8+ T cells in BCG-immunized C57BL/6 but not in BCG-immunized BALB/c mice, where such responses were weak and sporadic, reflecting the well-established Th1 bias of C57BL/6 mice vs. the Th2 bias of BALB/c mice. Similarly, BCG immunization alone has been found to induce a greater Th1 type response in C57BL/6 than BALB/c mice (43, 44), although this has not been observed universally (40). In any case, despite the disparate immune responses induced by the rLm5Ag (30) vaccine in BCG-immunized C57BL/6 and BALB/c mice, rLm vaccines expressing these five antigens boosted protection against Mtb aerosol challenge in both mouse strains. This result mirrors a previous observation that differences in the ability of these two mouse strains to generate Th1 helper cells are not reflected by differences in their ability to resist Mtb infection (45).

A potential major advantage of a Listeria-vectored vaccine, particularly with respect to protein/adjuvant vaccines, is enhanced capacity to induce CD8+ T cells. CD8+ T cells are required to resist Mtb infection, as demonstrated by studies in mice employing antibody-depletion or TAP1 knock-out of CD8+ T cells (46-49). Consistent with these observations, adoptive transfer of CD8+ T cells enhances resistance to TB (50). Of note, CD8+ T cells appear to play a more important role in primates than in rodents (51); hence, efficacy studies in rodents may underestimate the efficacy of Listeria-vectored vaccines in non-human primates and humans. In current studies, we are evaluating the efficacy of a multi-antigenic Listeria-vectored vaccine in non-human primates.

### Example 2: Listeria-Vectored Multi-Antigenic

Tuberculosis Vaccine Protects C57BL/6 and BALB/C Mice and Guinea Pigs Against Mycobacterium Tuberculosis Challenge

Aspects and embodiments of the invention discussed in this example are also discussed in Jia Q, Masleša-Galić S, Nava S, Horwitz MA. Commun Biol. 2022 Dec. 20; 5 (1): 1388. doi: 10.1038/s42003-022-04345-1 (termed in this example: “Jia et al.”), the contents of which are incorporated herein by reference.

Embodiments of the TB vaccines disclosed herein comprise a live attenuated replicating Listeria monocytogenes (Lm) bacterium expressing immunoprotective Mtb antigens. The Lm vector was chosen in large part because of its ability to induce robust antigen-specific CD4+ and CD8+ T cell responses to expressed recombinant antigens-both types of T cell immunity are central to immunoprotection against Mtb. The wild-type parent of this vector, a fast growing, Gram-positive, facultative intracellular bacterium that occasionally infects humans and can cause food-borne disease outbreaks, shares important features of its intracellular lifestyle with Mtb. Like Mtb, upon entry into host cells, which include mononuclear phagocytes, Lm initially resides in a phagosome, a site favoring antigen presentation via class II MHC molecules and the induction of antigen-specific CD4+ T cells. Subsequently, also in common with Mtb, Lm escapes the phagosome and multiplies in the host cytosol, a site favoring antigen presentation via class I MHC molecules and the induction of antigen-specific CD8+ T cells. As a result of its capacity to induce long-lived cell-mediated immune responses, genetically attenuated Lm has been developed as a vaccine vector for cancer and infectious diseases²⁴.

The specific Lm vector that we employ, Lm ΔactA ΔinlB prfA* (Lm ΔactA ΔinlB ΔuvrAB prfA*)^{22,25} has been attenuated from wild-type Lm and rendered more effective as a vaccine vector via several genetic manipulations²⁵. First, actA, a gene encoding the cell surface transmembrane protein ActA, which promotes intracellular motility via actin polymerization, has been deleted. ActA deletional mutants are able to grow within the cytosol of infected cells, but are unable to induce cell-to-cell spread, resulting in ˜1000-fold attenuation in virulence in mouse models²⁴. Second, a deletion of inlB, encoding internalin B, a virulence factor that promotes invasion of various mammalian cells including epithelial cells, endothelial cells and hepatocytes, inhibits Lm uptake into non-phagocytic cells, such as hepatocytes, but not into phagocytic cells, including antigen-presenting cells; hence, in the double deletional ΔactA ΔinlB mutant, off-target toxicity is minimized but not antigen presentation of secreted recombinant antigens in antigen presenting cells²⁶. Third, a point mutation (G155S) in the master virulence regulator PrfA that renders it constitutively active, promotes Lm escape into the host cell cytosol, and as a result of upregulated expression of PrfA and PrfA-dependent genes, shows enhanced expression of downstream recombinant proteins^{25,27,28}.

Using Lm ΔactA ΔinlB prfA* as a vaccine vector, we have developed several recombinant Lm-vectored Mtb vaccines (rLm) including rLm30²², rLmMtb5Ag (rLm5Ag)²³, and in this study, rLmMtb9Ag (rLm9Ag). The rLm30 vaccine expresses a single Mtb antigen—the 30-kDa major secretory protein or Antigen 85B (r30 or Ag85B, gene Rv1886); rLm5Ag expresses a fusion protein of 5 Mtb antigens—Mpt64/23.5 (Rv1980c), EsxH/TB10.4 (Rv0288), EsxA/ESAT6 (Rv3875), EsxB/CFP10 (Rv3874) and r30; and rLm9Ag expresses, in addition to the 5 antigens expressed by rLm5Ag, a fusion protein comprising 4 additional Mtb antigens-EspA (Rv3616c), EsxN (Rv1793), PPE68 (Rv3873) and TB8.4 (Rv1174c). These 4 additional proteins were selected on the basis of their capacity, when administered as part of a rLm booster vaccine, to enhance protective immunity against Mtb aerosol challenge in BCG-immunized mice²³. In previous studies of rLm30 and rLm5Ag, we have shown that i) immunization of mice with BCG has no significant effect on local replication or systemic dissemination, growth, and clearance of rLm30 administered intradermally 12 or 15 weeks later^{22,23}; ii) boosting BCG-primed mice with rLm30 and rLm5Ag enhances Mtb antigen-specific CD4+ and CD8+ T cell-mediated immune responses^{22,23}; and iii) boosting BCG-primed C57BL/6 and BALB/c mice with rLm30 and rLm5Ag enhances protective immunity against aerosolized Mtb^{22,23}.

Herein, we investigate the immunogenicity and efficacy of rLm5Ag and rLm9Ag as standalone vaccines in three animal models of pulmonary TB-inbred C57BL/6 and BALB/c mice and outbred Hartley guinea pigs. We test them as standalone vaccines—not because we envision them as replacement vaccines for BCG but because, as noted above, most of the people in the world in need of a TB booster vaccine were vaccinated with BCG in infancy; hence, their BCG-induced immunity will have largely waned by the time they would receive a TB booster vaccine many years and often decades later. As testing the potency of a heterologous booster vaccine administered decades after a prime vaccine is not feasible in small animal models, we elected instead to test the vaccines as standalone vaccines so as to mimic the situation in which BCG-induced immunity has completely waned. In so doing, we examined two different strains of mice, C57BL/6 and BALB/c, because these mice display different innate and acquired immune responses to infection, including mycobacterial infection with BCG 29,30 We additionally examined guinea pigs because these animals develop disease more akin to that of humans than do most strains of mice; e.g., they are highly susceptible to clinical disease after low dose infection with M. tuberculosis; they show strong cutaneous delayed-type hypersensitivity to tuberculin; and they display Langhans giant cells in lung lesions and develop caseating granulomas³¹.

In C57BL/6 and BALB/c mice, we show that homologous priming-boosting with rLm5Ag and rLm9Ag vaccines induces antigen-specific CD4+ and CD8+ T cell immune responses and protective immunity against aerosol challenge with virulent Mtb. In guinea pigs, we show that homologous priming-boosting with rLm9Ag induces Mtb antigen-specific lymphocyte proliferation and elevated frequencies of CD8+ T cells in the lungs and/or spleens, and that immunization with rLm5Ag or rLm9Ag induces significant protective immunity against Mtb aerosol challenge.

### Results

Construction and Verification of rLm9Ag Expressing Fusion Proteins of Mtb5Ag and Mtb5AgII from the comK and tRNAarg Loci, Respectively

To construct rLm5Ag (expressing Mtb ActAN-Mpt64-EsxH-EsxA-EsxB-r30)²³ (Supplementary Table 1 of Jia et al.) and rLm5AgII (expressing ActA-Mpt64-EsxN-PPE68-EspA-TB8.4) (Supplementary Table 1 of Jia et al.), we integrated the pPL2e-ActAN-Mtb5Ag and pPL2e-ActAN-Mtb5AgII into the tRNA^{arg} locus of the rLm chromosome (FIG. 1a of Jia et al., top and middle vaccines). To construct the rLm9Ag vaccine candidate (Supplementary Table 1 of Jia et al.), we integrated pPL1-ActAN-Mtb5Ag at the comK locus followed by integrating pPL2e-ActAN5AgII into the tRNA^{arg} locus of the Lm chromosome (FIG. 1a of Jia et al., bottom vaccine). This strategy had two advantages. First, the use of two expression cassettes allowed expression of smaller fusion proteins than a single cassette expressing all 9 antigens (estimated Mw of 9-antigen fusion protein of 212 kDa vs. the estimated molecular weights of the 5Ag and 5AgII fusion proteins of 94 and 118 kDa, respectively). The smaller Mw favors better stability and expression. Second, the strategy allows use of the same leader protein of Mpt64 for both the 5AgI and 5AgII expression cassette. We have tested various combinations of Mtb fusion proteins and found that Mtb proteins fused to the C-terminus of Mpt64 tend to be expressed more abundantly than otherwise; hence Mpt64 is evidently an ideal leader protein for the expression cassette of Mtb fusion proteins. We have shown that rLm5Ag, rLm5AgII, and two clones (clones 1 and 3) of rLm9Ag express the expected fusion proteins when grown in broth medium and in infected murine macrophage-like cells. As shown in FIG. 1b and Supplementary FIG. 1 left panels of Jia et al., rLm9Ag vaccine clones 1 and 3 (top panel, lanes 5, 6) express both the 94-kDa 5Ag and the 118-kDa 5AgII fusion proteins (indicated by arrows), similar to the proteins expressed by rLm5AgII (lane 4) and rLm5Ag (lanes 2, 3), respectively, that are detected by the polyclonal antibody to the ActA peptide. As expected, the 94-kDa, but not the 118-kDa, protein band was also detected by the polyclonal antibody to EsxH (middle panel, lanes 2, 3, 5, and 6) which is present in the 5Ag but not the 5AgII fusion protein.

To analyze expression of the heterologous Mtb fusion proteins by rLm growing inside of macrophages, we infected murine macrophage-like cells (J774.A1) with LmVector or rLm expressing various Mtb fusion proteins at a Multiplicity of Infection (MOI) of 10. At 5.5 hours post infection, we harvested the infected cells and analyzed the lysates for protein expression by SDS-PAGE and Western blotting using a polyclonal antibody to a N-terminal peptide comprising 18 amino acids (A30-K47) of ActA (AK18) (courtesy of Justin Skoble and Peter Lauer), which detected the predicted 94-kDa (5Ag) protein band (and non-specific protein bands) from J774A.1 cells infected with rLm5Ag, rLm5AgII, or rLm9Ag clones #1 and 3 (FIG. 1c upper panel and Supplementary FIG. 1 right upper panel of Jia et al., lanes 2, 3, 5, and 6, respectively) and the predicted 112-kDa (5AgII) protein band from J774A.1 cells infected with rLm5Ag I and rLm9Ag clones #1 and 3 (FIG. 1c of Jia et al., upper panel, lanes 4, 5, and 6, respectively), but not from LmVector-infected (upper panel, lane 1) and mock infected (upper panel, lane 7) J774A.1 cells. Antibody to Lm P60 detected a ˜60-kDa protein band from Lm-infected (FIG. 1c lower panel and Supplementary FIG. 1 right lower panel of Jia et al., lanes 1-6), but not from mock infected cells (FIG. 1c of Jia et al., lower panel, lane 7). Antibody to β-actin detected a ˜42 kDa band from uninfected and infected J774 cell lysates, as expected (FIG. 1c of Jia et al. lower panel, and Supplementary FIG. 1 of Jia et al. right lower panel, lanes 1-7).

Genetic Stability and Growth Kinetics of rLm5Ag and rLm9Ag Vaccines

To evaluate the antigen expression cassette stability of rLm grown in broth culture and in infected macrophage-like cells, we examined the growth of rLm vaccine candidates in BHI broth supplemented with various antibiotics and in infected monolayers of the J774A.1 cells with stationary grown rLm vaccines and assayed bacterial replication. As shown in Supplementary FIG. 2 of Jia et al., the rLm9Ag, rLm5Ag, and rLm5Ag I vaccine candidates grew similarly in the presence or absence of antibiotic selection, either after direct inoculation into BHI broth medium (Supplementary FIG. 2a of Jia et al.) or after passage in murine macrophage-like cells followed by plating onto BHI agar plates supplemented with various antibiotics (Supplementary FIGS. 2b & 2c of Jia et al.). We verified the heterologous and homologous protein expression by Western blotting as shown in FIG. 1b and FIG. 1c of Jia et al. In addition, we passaged LmVector, rLm5Ag and rLm9Ag vaccines daily for consecutive days on BHI agar plates and verified the stability of the antigen expression cassettes. We also verified the stability of the Mtb antigen expression cassettes after the rLm9Ag vaccine was passaged in guinea pigs (Supplementary FIG. 3 of Jia et al.). Thus, the antigen expression cassettes for Mtb 5Ag (Mpt64-EsxH-EsxA-EsxB-r30), Mtb 5AgII (Mpt64-EsxN-PPE68-EspA-TB8.4), and Mtb 9Ag (5Ag+5AgII) are stably maintained in the rLm vaccine candidates.

To examine the growth kinetics of rLm vaccine candidates in murine macrophages, we infected monolayers of J774A.1 cells with LmVector or rLm vaccines at an MOI of 10, as described in the legend to Supplementary FIG. 2d of Jia et al. In general, all rLm5Ag, rLm5AgI and rLm9Ag vaccines grew similarly to LmVector at 2 h post infection; at 4 h post infection, there were some delays in the growth of rLm5AgII and rLm9Ag clone #1 compared with LmVector; at 6 hours post infection, there were delays in the growth of rLm5AgII and rLm9Ag clone #1 and clone #3; rLm5Ag grew similarly to LmVector at 2, 4, and 6 h post infection (Supplementary FIG. 2d of Jia et al.). These results suggest that the fusion protein expression cassettes for Mtb 5AgII imposed a modest burden on its bacterial host that caused some growth delay in macrophages.

rLm5Ag Induces Antigen-Specific T-Cell Mediated Immune Responses in Mice

We examined the capacity of rLm5Ag to induce antigen-specific T cells and cytokine-expressing CD4+ and CD8+ T cells in the lungs and spleens of C57BL/6 mice and BALB/c mice (FIG. 2-5, Supplementary FIGS. 4-7 of Jia et al.). C57BL/6 mice immunized with rLm5Ag or LmVector produced comparable frequencies of CD3+ T cells among gated lymphocytes and comparable frequencies of CD4+, CD8+ and CD4-CD8-T cells among CD3+ T cells after 22 h antigen stimulation in the lungs, and for the most part in the spleens, although differences in response to a few antigens were statistically significant (Supplementary FIG. 5 of Jia et al.).

With respect to antigen-specific cytokine-expressing T cells, the rLm5Ag-immunized C57BL/6 mice produced significantly greater frequencies of cytokine-producing CD4+ T cells in their lungs and spleens expressing IFN-γ, TNF-α, and/or IL2 and polyfunctional CD4+ T cells expressing two or more cytokines among IFN-γ, TNF-α, and IL-2 in response to in vitro 6 h stimulation with Ag85B (FIG. 2e, 2m, 3a, 3e of Jia et al.), 5Ag pool (FIG. 2f, 2n, 3b, 3f of Jia et al.), or PPD (FIG. 2g, 20, 3c, 3g of Jia et al.) than mice immunized with LmVector. No significant differences in the frequencies of CD4+ T cells expressing any of the cytokines were detected after in vitro stimulation with 23.5/Mpt64 (FIG. 2a, 2i of Jia et al.), TB10.4/EsxH (FIG. 2b, 2j of Jia et al.), ESAT6/EsxA (FIG. 2c, 2k of Jia et al.), or CFP10/EsxB (FIG. 2d, 21 of Jia et al.), or as expected PMA (FIG. 2h, 2p of Jia et al.). Notably, mice immunized with rLm5Ag also produced greater frequencies of CD8+ T cells expressing IFN-γ and TNF-α and polyfunctional CD8+ T cells expressing both cytokines in response to in vitro stimulation with 23.5/Mpt64 (FIG. 4a, 5a of Jia et al.), TB10.4/EsxH (FIG. 4b, 5b of Jia et al.), 5Ag pool (FIG. 4f, 5c of Jia et al.), and PPD (FIG. 4g, 5d of Jia et al.) than mice immunized with LmVector in the lung, and a greater frequency of CD8+ T cells expressing TNF-α in response to the 5Ag pool in the spleen (FIG. 4n of Jia et al.). Of note, the frequencies of antigen specific cytokine-producing CD4+ and CD8+ T cells in the lungs and spleens after 22-hour antigen stimulation were similar to those after 6-hour stimulation. Thus, homologous priming-boosting C57BL/6 mice with rLm5Ag induces Mtb antigen-specific cytokine-expressing CD4+ and CD8+ T cells, especially Ag85B-specific CD4+ T cells expressing IFN-γ, TNF-α and IL-2, and Mpt64 and EsxH-specific CD8+ T cells expressing IFN-γ and TNF-α.

BALB/c mice immunized three times with rLm5Ag produced substantially greater (˜2-5-fold) frequencies of live lung CD3+ T cells than LmVector-immunized mice after 22h incubation whether incubated with or without antigen (P<0.05-P<0.001) (Supplementary FIG. 6a of Jia et al.); of these greatly expanded numbers of CD3+ T cells, CD4+ T cell frequencies were somewhat reduced and CD8+ and CD4-CD8-T cell frequencies were somewhat increased, sometimes significantly so, whether incubated with or without antigen (P<0.001-P<0.0001 without antigen) (Supplementary FIG. 6b, c, d of Jia et al.). In contrast, in the spleen, frequencies of CD3+, CD4+, CD8+, and CD4⁻ CD8⁻ T cells were generally comparable (Supplementary FIG. 6e-h of Jia et al.).

With respect to antigen-specific cytokine-expressing CD4+ T cells, in the spleens, BALB/c mice immunized with rLm5Ag produced significantly greater amounts of IFN-γ, TNF-α, and/or IL-17A expressing CD4+ T cells in response to 6h in vitro stimulation with Mpt64, EsxH, EsxB, Ag85B, PPD and GI-H37RV than mice immunized with LmVector; the only antigens not inducing a significantly greater response was EsxA and, as expected, PMA (Supplementary FIG. 7 of Jia et al., top two rows, CD4+ T cells). In contrast, with respect to antigen-specific cytokine-expressing CD8+ T cells, BALB/c mice immunized with rLm5Ag produced moderately greater frequencies of splenic CD8+ T cells expressing only IL-17A or IL-17A and IFN-γ in response to only EsxH and EsxA but not to Mpt64, EsxB, Ag85B, PPD, GI-H37RV and, as expected, PMA (Supplementary FIG. 7 of Jia et al., bottom two rows, CD8+ T cells).

Thus, homologous priming-boosting BALB/c mice with rLm5Ag induces Mtb antigen-specific cytokine-expressing CD4+ and CD8+ T cells, where the CD4+ T cells show specificity to Mpt64, EsxH, EsxB, and Ag85B—all but EsxA—and express predominantly IFN-γ and TNF-α, and CD8+ T cells show specificity to EsxH and EsxA, but express predominantly IL-17A and IFN-γ.

Comparing C57BL/6 and BALB/c mice, immunization with rLm5Ag substantially increases the frequencies of live CD3+ T cells in the lungs of BALB/c mice (Supplementary FIG. 6a of Jia et al.) but not C57BL/6 mice (Supplementary FIG. 5a of Jia et al.) independent of antigen. Both mice produce antigen-specific cytokine-expressing CD4+ and CD8+ T cells, although the antigen specificity and suite of cytokines secreted differ between these two mouse strains.

rLm5Ag and rLm9Ag Induce Protective Immunity Against Aerosol Challenge with Virulent Mtb Erdman Strain in BALB/c and C57BL/6 Mice

In preliminary studies, we evaluated the protective efficacy in C57BL/6 and BALB/c mice of a combination of two rLm vaccines expressing 5 antigens-r30/Ag85B, 23.5/Mpt64, TB10.4/EsxH, ESAT6/EsxA, and CFP10/EsxB (combination of rLm30+rLm4Ag, designated as rLm5Ag*). First, we explored i.d. and intranasal (i.n.) administration of the composite vaccine rLm5Ag* in C57BL/6 mice. In these experiments, we used BCG as a positive control against which to compare the efficacy of the Lm vaccines, as BCG consistently provides strong efficacy in animal models of TB. We immunized groups of C57BL/6 mice, 8 per group, with PBS or BCG i.d. or i.n. at Week 0, or the rLm5Ag* i.d. or i.n. three times at Weeks 0, 7, and 10, challenged all the mice with aerosolized Mtb (average of 24 CFU of the Mtb Erdman strain delivered to the lungs of each mouse, as assayed on Day 1 post-challenge) at Week 13 and euthanized the mice at Week 23 (FIG. 6a of Jia et al., top panel). Mice immunized i.d. three times with rLm5Ag* had significantly lower CFUs in their lungs and spleens than Sham-immunized mice (P<0.05 and P<0.01, respectively), not significantly different from mice immunized i.d. once with BCG (FIG. 6a of Jia et al., left middle and bottom panels); mice immunized i.n. once with BCG or three times with rLm5Ag* showed no reduction in bacillus burden in the lungs compared with Sham-immunized mice (FIG. 6a of Jia et al., right middle panel), but had significantly lower CFUs in their spleens than Sham-immunized mice (P<0.0001 and P<0.05, respectively) (FIG. 6a of Jia et al., right bottom panel).

Next, we immunized groups of 8 BALB/c mice with PBS (Sham) or BCG (positive control) i.d. at Week 0, or i.m. with the rLm5Ag* three times at Weeks 10, 14, and 18 or twice at Weeks 14 and 18, then challenged the mice at Week 22 with aerosolized Mtb (average of 30 CFU of the Mtb Erdman strain delivered to the lungs of each mouse, as assayed on Day 1 post-challenge), and euthanized the mice 10 weeks later (Week 32) to assay bacillus burden in their lungs and spleens (FIG. 6b of Jia et al., upper panel). As shown in FIG. 6b of Jia et al., middle and bottom panels, mice immunized i.m. twice or three times with rLm5Ag* had significantly lower CFUs in their lungs and spleens than Sham-immunized mice; the reduction in CFU in the lungs was comparable to that of mice immunized with BCG. Three immunizations were slightly more efficacious than two immunizations and resulted in a greater statistically significant difference from sham in both the lung and spleen.

Subsequently, we performed definitive studies comparing the protective efficacy of rLm30, rLm5Ag* (combination of rLm30+rLm4Ag), rLm5Ag (single vaccine expressing the same 5 antigens as rLm5Ag* from the tRNA^{arg} locus), and rLm9Ag (clones #1 and #3) as a standalone vaccine in both C57BL/6 and BALB/c mice immunized three times s.q., a route that was found to be both practical and efficacious against Mtb aerosol challenge in heterologous prime-boost studies involving a BCG prime and an rLm boost. We immunized groups of 8 C57BL/6 and BALB/c mice s.q. three times at Weeks 0, 3, and 6 with rLm30, rLm5Ag*, rLm5Ag, or two individual clones of rLm9Ag (rLm9Ag #1 and rLm9Ag #3); unimmunized (UI) mice or mice immunized i.d. with BCG, or three times s.q. with LmVector served as controls. Four weeks after the last immunization, mice were challenged with aerosolized Mtb (average of 10 CFU of Mtb Erdman strain delivered to the lungs of each mouse, as assayed at Day 1 post challenge). At Week 20, the mice were euthanized and organ bacillus burdens assayed (FIGS. 7a and 8a of Jia et al.).

As shown in FIG. 7 of Jia et al., C57BL/6 mice immunized with BCG once or immunized three times with rLm30, rLm5Ag*, rLm5Ag, and both clone #1 and #3 of rLm9Ag, had lower CFUs in their lungs and spleens than sham-immunized mice and mice immunized with LmVector. Among the 4 rLm vaccines tested, rLm9Ag provided the greatest protection to C57BL/6 mice against Mtb aerosol challenge. Specifically, in the lung, mice immunized with both clones #1 and #3 of rLm9Ag had significantly lower CFU then sham-immunized mice (−0.4 and −0.5 logs, respectively (P<0.05) and mice immunized with LmVector (P<0.01), comparable to mice immunized with BCG (FIG. 7b of Jia et al., left panel). In the spleen, mice immunized with clone #1 of rLm9Ag had significantly lower CFU (−0.7 log) then sham-immunized mice (P<0.05). Mice immunized with clone #3 of rLm9Ag had significantly lower CFU than both sham-immunized mice (−0.8 logs) (P<0.05) and mice immunized with LmVector (P<0.05), similar to and not statistically significantly different from mice immunized with BCG (FIG. 7b of Jia et al., right panel). These results indicate that vaccinating C57BL/6 mice with rLm vaccines comprising 5 antigens or 9 antigens as a standalone vaccine provides potent protection against aerosol challenge with virulent Mtb Erdman strain and the rLm9Ag vaccine provides protection comparable to BCG.

As shown in FIG. 8 of Jia et al., BALB/c mice immunized with BCG once or immunized three times with rLm30, rLm5Ag*, rLm5Ag, or both clones #1 and #3 of rLm9Ag had lower CFUs in their lungs and spleens than unimmunized mice and mice immunized with LmVector. Among the 5 rLm vaccine candidates tested, rLm5Ag* (combination of rLm30+rLm4Ag) and rLm5Ag (single vaccine) provided the best protection to BALB/c mice against Mtb aerosol challenge. Specifically, in the lung, mice immunized with rLm5Ag* and rLm5Ag had significantly lower CFU then sham-immunized mice (−1.1 and −1.1 logs, respectively) (P<0.0001 and P<0.0001) and mice immunized with LmVector (P<0.0001 and P<0.01), comparable (difference not statistically significant) to mice immunized with BCG. Mice immunized with rLm9Ag #1 and rLm9Ag #3 also had significantly lower CFU then sham-immunized mice (−0.8 and −0.9 logs, respectively) and mice immunized with LmVector (P<0.01 and P<0.001) in the lung (FIG. 8b of Jia et al., left panel). In the spleen, mice immunized with rLm5Ag* and rLm5Ag had significantly lower CFU than sham-immunized mice (−1.6 and −1.7 logs, respectively) (P<0.0001 and P<0.0001) and LmVector (−1.1 and −1.2 logs, respectively) (P<0.001 and P<0.0001). Mice immunized with rLm9Ag clone #1 and #3 also had significantly lower CFU then unimmunized mice (−0.9 and −1.1 logs, respectively) (P<0.0001 and P<0.0001) and mice immunized with LmVector (−0.3 and −0.6 logs, respectively) (P<0.001 and P<0.0001) (FIG. 8b of Jia et al., right panel). These results indicate that in BALB/c mice, vaccinating with rLm5Ag*, rLm5Ag, or rLm9Ag as a standalone vaccine provides potent protection against aerosol challenge with virulent Mtb Erdman strain.

rLm9Ag Induces Antigen Specific T-Cell Proliferation in Guinea Pigs

To evaluate the capacity of the rLm9Ag vaccine to induce T-cell mediated immune responses in guinea pigs, we immunized guinea pigs with rLm9Ag or LmVector three times at Weeks 0, 3, and 6 and 6 days later evaluated responses of spleen and lung lymphocytes, CD4+ T cells, and CD8+ T cells to stimulation with Mtb peptide antigens. As shown in FIG. 9 of Jia et al., in general, lung (FIG. 9a of Jia et al.) and spleen (FIG. 9b of Jia et al.) cells of guinea pigs immunized with rLm9Ag had greater frequencies of lymphocytes than guinea pigs immunized with LmVector after antigen stimulation. Specifically, in the lungs, guinea pigs immunized with rLm9Ag produced greater frequencies of lymphocytes in response to Ag85B, EspA, EsxA, EsxB, EsxH, EsxN, PE68, TB8.4 and PPD (FIG. 9a of Jia et al.). In the spleens, guinea pigs immunized with rLm9Ag produced greater frequencies of lymphocytes in response to Ag85B, EsxA, EsxB, and TB8.4 (FIG. 9b of Jia et al.). As expected, there were no significant differences between animals immunized with LmVector and rLm9Ag in frequencies of lymphocytes stimulated without antigen (Medium alone) or with ConA (positive control) (FIG. 9a, 9b of Jia et al.). With respect to proliferating CD4+ T cells (CD4+CTVLow) in the lung, as shown in FIG. 9c of Jia et al., no significant differences were detected between guinea pigs immunized with LmVector and rLm9Ag after in vitro stimulation with Mtb peptide antigens. With respect to proliferating CD4+ T cells (CD4+CTVLow) in the spleen, as shown in FIG. 9d of Jia et al., guinea pigs immunized with rLm9Ag had greater frequencies of proliferating CD4+ T cells to Ag85B and EsxB than guinea pigs immunized with LmVector. With respect to proliferating CD8+ T cells (CD8+CTVLow) in the lung, as shown in FIG. 9e of Jia et al., there were no significant differences between guinea pigs immunized with LmVector and rLm9Ag after in vitro stimulation with Mtb peptide antigens. With respect to proliferating CD8+ T cells (CD8+CTVLow) in the spleen, as shown in FIG. 9f of Jia et al., guinea pigs immunized with rLm9Ag had greater frequencies of CD8+ T cells in response to Ag85B, EsxB, EsxN, PPE68, TB8.4, and PPD than guinea pigs immunized with LmVector.

Thus, rLm9Ag induces significantly increased lung and/or spleen cells in response to 8 of its 9 recombinant Mtb antigens, significantly increased proliferating splenic CD4+ T cells in response to Ag85B and EsxB, and significantly increased proliferating CD8+ T cells in response to 5 of its 9 Mtb antigens.

rLm5Ag and rLm9Ag Induce Protective Immunity Against Aerosol Challenge with Virulent Mtb Erdman Strain in Outbred Guinea Pigs

Finally, we evaluated the capacity of the rLm5Ag and rLm9Ag vaccines to induce protective immunity against Mtb aerosol challenge in the outbred guinea pig model. As shown in FIG. 10a of Jia et al., we immunized guinea pigs three times s.q. at Weeks 0, 3, and 6 with the rLm multi-antigenic vaccines or LmVector, challenged them at Week 10 with aerosolized Mtb Erdman strain, subsequently monitored their weight for 10 weeks, and then euthanized them to determine CFU in their lungs and spleens; guinea pigs immunized with BCG ID served as a positive control. As shown in FIG. 10b, Supplementary FIG. 8 and Supplementary Table 2 of Jia et al., guinea pigs immunized three times at Weeks 0, 3, and 6 with rLm9Ag at low, medium, and high doses, or with rLm5Ag at a medium dose gained significantly more weight 4-10 weeks post-challenge than guinea pigs immunized with LmVector (P<0.05-P<0.0001); the weight gain of rLm vaccinated guinea pigs was not significantly different from that of guinea pigs immunized with BCG. Similarly, as shown in FIG. 10c of Jia et al., guinea pigs immunized with low, medium, or high doses of rLm9Ag had lower Mtb bacillus burdens than guinea pigs immunized with LmVector in their lungs (0.2, 0.8, and 0.4 logs lower, respectively) (FIG. 10c of Jia et al., left panel) and spleens (0.2, 1.2 and 0.9 logs lower, respectively) (FIG. 10c of Jia et al. right panel). Similarly, guinea pigs immunized with rLm5Ag also had lower bacterial burdens than LmVector immunized guinea pigs in the lung and spleen (0.8 and 0.9 logs lower, respectively), reductions virtually equivalent to those in animals immunized with the medium dose of rLm9Ag. Guinea pigs immunized with BCG also had significantly lower Mtb bacillus burdens in their lungs (FIG. 10c of Jia et al. left panel) (1.4 logs lower) and spleens (FIG. 10c of Jia et al. right panel) (2.2 logs lower) than those immunized with LmVector. Among the 3 doses of rLm9Ag tested, the medium dose (10⁶) of rLm9Ag provided the best protection against aerosol challenge with highly virulent Mtb. Thus, both the rLm5Ag and rLm9Ag vaccines induce potent protective immunity in guinea pigs.

### DISCUSSION

Our studies show that homologous priming-boosting of inbred C57BL/6 and BALB/c mice and outbred guinea pigs with rLm5Ag (expressing a fusion protein of Mtb antigens Mpt64-EsxH-EsxA-EsxB-Ag85B) and rLm9Ag (additionally expressing Mtb antigens Mpt64-EsxN-PPE68-EspA-TB8.4) induces antigen-specific CD4+ and CD8+ T-cell mediated immunity and immunoprotection against aerosol challenge with virulent Mtb Erdman in all three animal models.

Both CD4+ and CD8+ T cells are required to control primary TB infection. CD4+ T cells help CD8+ T cells maintain effector function and prevent exhaustion, and the synergy between CD4+ and CD8+ T cells promotes the survival of mice infected with Mtb 32. Our results show that rLm5Ag induces both CD4+ and CD8+ T cell-mediated immune responses in both C57BL/6 and BALB/c mice. In both mouse models, immunizing with rLm5Ag or rLm9Ag induces elevated frequencies of lung and/or splenic CD8+ T cells, consistent with Lm's reputation as a potent inducer of CD8+ T cells 24. Similarly, in guinea pigs, rLm9Ag induces proliferating antigen-specific CD4+ and CD8+ T cells. CD8+ T cells appear to play a more important role in protection against Mtb in primates than in rodents 33. Hence, studies in rodents may underestimate the efficacy of an rLm vaccine in non-human primates and humans.

In C57BL/6 mice, rLm5Ag induced significantly elevated frequencies of antigen-specific polyfunctional CD4+ and CD8+ T cells expressing IFN-γ, TNF-α, and sometimes IL-2 (the CD4+ T cells in response to stimulation with Ag85B, and the CD8+ T cells in response to stimulation with Mpt64 and EsxH); the frequency of IL-17A expressing cells was not significantly elevated. In BALB/c mice, rLm5Ag induced significantly elevated frequencies of CD4+ T cells expressing IFN-γ and TNF-α (in response to stimulation with Mpt64, EsxH, EsxB, and Ag85B), and additionally significantly elevated frequencies of CD4+ and CD8+ T cells secreting IL17A (in response to EsxH and EsxA). Hence, these two mouse strains displayed a somewhat different CD4+ and CD8+ T cell cytokine expression profile after rLm5Ag immunization.

Among the five antigens common to both rLm5Ag and rLm9Ag, all five induced T cell responses in at least one animal model, and four induced T cell responses in multiple animal models. Antigen 85B was an especially dominant antigen, inducing T cell responses in all three animal models-cytokine-expressing CD4+ T cells in both C57BL/6 and BALB/c mice and proliferating CD4+ and CD8+ T cells in guinea pigs. EsxB also stood out, inducing cytokine-expressing CD4+ T cells in BALB/c mice and proliferating CD4+ and CD8+ T cells in guinea pigs. EsxH induced cytokine-expressing CD4+ T cells in both C57BL/6 and BALB/c mice. Finally, Mpt64 induced cytokine-expressing CD4+ T cells in BALB/c mice and CD8+ T cells in C57BL/6 mice. The rLm9 Ag vaccine, containing 4 additional Mtb antigens, was tested for immunogenicity only in guinea pigs. Three of these four new antigens-EsxN, PPE68, and TB8.4-induced proliferating CD8+ T cells in guinea pigs.

An Lm-vectored vaccine has major advantages as a TB vaccine including i) Lm multiplies rapidly intracellularly and secrets foreign antigens into the host cell cytosol, as noted above, and then is rapidly cleared-7 to 10 days post immunization²²; ii) Lm-vectored vaccines with ΔactA ΔinlB deletions have an established safety profile in humans; the vaccines were well tolerated in a Phase I study³⁴; iii) pre-existing immunity to Lm^{35,36} and to BCG does not deleteriously affect immunization with Lm-vectored vaccines 22, in contrast to some virus- and mycobacterium-vectored vaccines; iv) Lm-vectored vaccines have enhanced capacity to induce both CD4+ and CD8+ T cell-mediated immune responses, and is an especially potent inducer of CD8+ T cells, as noted above; and v) Lm-vectored vaccines can be cheaply manufactured in broth medium at large scale without the need for extensive purification as with protein/adjuvant vaccines or virus-vectored vaccines grown in mammalian cells.

Although in this study we tested the Listeria vectored vaccines as standalone vaccines, we envision an rLm vaccine not as replacement vaccine for BCG but as a heterologous booster vaccine for people previously vaccinated with BCG, or in the future with an improved mycobacterial vaccine that eventually replaces BCG. Greater than 5 billion people on earth who have been vaccinated with BCG live in TB endemic areas, and hence might benefit from a heterologous TB booster vaccine. As most of these people, including adolescents and adults, would have been vaccinated with BCG in infancy, their BCG-induced immunity is likely to have largely waned by the time they were to receive such a booster vaccine many years and decades later. For this reason, as noted earlier, we considered it important to test the efficacy of our Listeria vectored vaccines as standalone vaccines. As standalone vaccines, we did not expect the rLm vaccines to be superior to BCG, which shares thousands of antigens with M. tuberculosis. However, that our studies demonstrate that the rLm vaccines are in many cases comparable in potency to BCG is noteworthy and, in our view, strongly supports the continued development of these rLm vaccines as TB booster vaccines. In conclusion, in this study, the protective efficacy of multi-antigenic rLm TB vaccines—including rLm5Ag and rLm9Ag—was demonstrated in three rigorous animal models of pulmonary TB. This follows upon the demonstration in previous studies of the protective efficacy of a single-antigen rLm vaccine (rLm30) and multi-antigenic rLm vaccines—including rLm5Ag—as booster vaccines to enhance the level of immune protection afforded by BCG immunization. Hence, an rLm vaccine expressing multiple Mtb immunoprotective antigens has substantial promise as a new vaccine to combat the TB pandemic.

### Material and Methods

### Ethics Statement

All animals were maintained in a specific-pathogen-free animal facility and used according to protocols approved by the UCLA Institutional Animal Care and Use committee.

### Cell Lines, Bacteria, Animals, Protein Antigens, and Antibodies.

Murine (J774A.1, ATCC TIB-67) monocytes were cultured as we described previously²². M. bovis BCG Tice and Mtb Erdman (ATCC 35801) strains were acquired and stocks prepared as we described previously²². The Listeria vector, Lm ΔactA ΔinlB prfA*^{22,25}, derived from Listeria monocytogenes 10403S strain (phage-cured, DP-L4056)³⁷, and recombinant Lm-vectored vaccines were grown to mid-log phase in Yeast Extract broth medium, collected by centrifugation, resuspended in PBS, titrated, and stored in 20% glycerol at −80° C. until use. Six to eight-week-old female C57BL/6 and BALB/c mice were purchased from Envigo (Indianapolis, IN) or Jackson Laboratory (Bar Harbor, Maine, USA) and three-week-old outbred male Hartley strain guinea pigs were purchased from Charles River Laboratories (Wilmington, MA, USA). The following Mtb protein reagents were obtained through BEI Resources, NIAID, NIH: Ag85B (Gene Rv1886c), Purified Native Protein from Strain H37Rv, NR-14857; ESAT-6, Recombinant Protein Reference Standard, NR-49424; CFP-10, Recombinant Protein Reference Standard, NR-49425; Mpt64, Recombinant Protein Reference Standard, NR-44102; and GI-H37RV, Mtb, Strain H37Rv, Gamma-Irradiated Whole Cells, NR14819. The Mtb protein EsxH/TB10.4 (gene Rv0288) was obtained from Aeras (formerly Rockville, Maryland, United States). Rabbit polyclonal antibody to ActAN (AK18, lot D4698) was obtained courtesy of Justin Skoble and Pete Lauer; rabbit polyclonal antibody to TB10.4 was obtained from Aeras (formerly Rockville, Maryland, United States); monoclonal antibody to Lm P60 (P6007, Lot AG-20A-0022-C100) was purchased from AdipoGen (San Diego, United States); and monoclonal antibody to β-actin (A5441) was purchased from Sigma (St. Louis, United States).

### Construction of Lm-Vectored Multi-Antigenic Vaccines.

We constructed Lm-vectored multi-antigenic rLm vaccine candidates using the Lm ΔactA ΔinlB prfA* vector²⁵ and two Lm site-specific phage integration vectors, pPL1 and pPL2, through conjugation process, as previously described by us and others^{22,23,37}. The pPL1 conjugation vector (kindly provided by Peter Lauer) utilizes the listeriophage U153 integrase and attachment site for insertion at the comK locus of the rLm chromosome and carries a Gram-positive chloramphenicol acetyltransferase gene; the pPL2e-derived conjugation vector (pBHE666 containing actA promoter and the N-terminal 100 amino acids of ActAN, kindly provided by Justin Skoble) modified from pPL2, utilizes the listeriophage PSA integrase and attachment site for insertion in the 3′ end of the tRNA^{arg} gene of the rLm chromosome and carries an erythromycin resistance gene³⁷. We cloned the genes encoding Mtb proteins, optimized for expression of Mtb proteins in Listeria monocytogenes and purchased from DNA2.0 (currently https://www.atum.bio/) (Newark, CA), into pPL1 and pBHE666 by the restriction enzyme method and in some cases by the Electra Vector System (https://www.atum.bio/) to generate the following plasmids: pPL2e-ActAN-Mtb4Ag (for integration of ActAN-Mtb4Ag expression cassette at the tRNA^{arg} locus to construct rLm4Ag), pPL2e-ActAN-Mtb5Ag (for integration of ActAN-Mtb5Ag expression cassette at the tRNA^{arg} locus to construct rLm5Ag), pPL1-ActAN-Mtb5Ag (for integration of ActAN-Mtb5Ag expression cassette at the comK locus to construct rLm9Ag), and pPL2e-ActAN-Mtb5AgII (for integration of Mtb5AgII expression cassette at the tRNA^{arg} locus to construct rLm9Ag). We have deposited the sequences of these plasmids to Genbank (https://www.ncbi.nlm.nih.gov/genbank/). All molecular plasmid constructs were confirmed by restriction enzyme digestion and nucleotide sequencing. Candidate vaccines rLm30 (expressing ActAN-r30/Ag85B) (Supplementary Table 1 of Jia et al.)²², rLm4Ag (expressing the fusion protein ActAN-Mpt64-EsxH-EsxA-EsxB) (Supplementary Table 1 of Jia et al.), and rLm5Ag (expressing the fusion protein ActAN-Mpt64-EsxH-EsxA-EsxB-r30) (Supplementary Table 1 of Jia et al.) were constructed previously²³ [where rLm5Ag is referred to as rLm5Ag (30)]; the Mtb protein expression cassettes in these vaccines were cloned into the pPL2e-derived vector and integrated at the tRNA^{arg} locus of the rLm chromosome. The rLm5AgII vaccine candidate (Supplementary Table 1 of Jia et al.), expressing the Mtb fusion protein of ActAN-Mpt64-EsxN-PPE68-EspA-TB8.4 from the pPL2e vector integrated at the tRNA^{arg} locus as well, was constructed similarly as described previously^{22,23}. The rLm9Ag vaccine candidate (Supplementary Table 1 of Jia et al.) was constructed by integrating the pPL1-ActAN-Mtb5Ag (expressing ActAN-Mpt64-EsxH-EsxA-EsxB-r30) at the comK locus followed by integrating the pPL2e-ActAN-5AgII (expressing ActAN-Mpt64-EsxN-PPE68-EspA-TB8.4) at the tRNA^{arg} locus. The resultant rLm9Ag carries a total of 9 Mtb antigens with Mpt64 being a common antigen located at the N-terminus of both 5Ag and 5AgII fusion proteins. The pPL1-ActAN-Mtb5Ag conjugation vector carries a codon-optimized antigen expression cassette for the fusion protein of Mpt64 (Δ1V-23A)-RP-EsxH-GGSG (SEQ ID NO: 3)-EsxA-GSSGGSSG (SEQ ID NO: 4)-EsxB-GSSGGSSG (SEQ ID NO: 4)-Ag85B (Δ2Q-43A) (abbreviated as Mpt64-EsxH-EsxA-EsxB-r30), in which RP is a dipeptide encoded by an EagI restriction enzyme site, and GSSG (SEQ ID NO: 6) and GSSGGSSG (SEQ ID NO: 4) are flexible fusion protein linkers. The pPL2e-ActAN-Mtb5AgII conjugation vector carries a codon-optimized antigen expression cassette for the fusion protein of Mpt64 (Δ1V-23A)-EsxN-GSSG (SEQ ID NO:)-PPE68-GSSGGSSG (SEQ ID NO: 4)-EspA (Δ111F-193L)-GSSGGSSG (SEQ ID NO: 4)-TB8.4 (Δ2R-28A) (abbreviated as Mpt64-EsxN-PPE68-EspA-TB8.4).

### Growth Kinetics and Stability of Lm-Vectored Multi-Antigenic Vaccines In Vitro

The growth kinetics of rLm in broth and in murine macrophage-like cells were evaluated as described previously by us^{22,23}. To assay the stability of rLm vaccines grown on agar plates, we passaged the Lm vector, rLm5Ag and rLm9Ag daily for 10 consecutive days on BHI agar plates supplemented with streptomycin, and at day 5 and day 10, we transferred 20-25 colonies of each vaccine onto BHI plates supplemented with streptomycin plus erythromycin (marker for antigen expression cassette integrated at the tRNA^{arg} locus) or streptomycin plus chloramphenicol (marker for antigen expression cassette integrated at the comK locus). To assay vaccine stability in macrophage-like cells, we infected monolayers of J774A.1 cells with rLm vaccines in the absence of antibiotic selection for 5.5 hours; lysed the cells; serially diluted the lysates and plated them on BHI agar supplemented with various antibiotics; cultured the plates at 37° C. for 2 days; and counted the colonies.

### Immunization of Mice and Intracellular Cytokine Staining of Mouse Spleen and Lung Cells.

To determine the immunogenicity of rLm5Ag as a standalone vaccine, we immunized C57BL/6 and BALB/c mice, 4/group, s.q. at Weeks 0, 4, and 8 with 2×10⁶ Colony Forming Units (CFU) of the Lm vector (LmVector) or rLm5Ag (expressing the fusion protein of ActAN-Mpt64-EsxH-EsxA-EsxB-r30 from the tRNA^{arg} locus); euthanized the mice at 6 days post the last immunization; prepared single cell suspensions of spleen and lung cells; stimulated the single cell suspensions with various Mtb antigens for 6 h or 22 h; and assayed T-cell immunity by intracellular cytokine staining (ICS) using an eight-color flow cytometry panel to analyze simultaneously multiple cytokines at the single-cell level as described by us previously^{22,23}.

Immunization and Aerosol Challenge of Mice with Virulent Mtb Erdman Strain.

Groups of BALB/c or C57BL/6 mice, 8/group, were vaccinated intradermally (i.d.), intramuscularly (i.m.), intranasally (i.n.), or s.q. two or three times, 3 or 4 weeks apart, with 10⁶ CFU of LmVector or of multi-antigenic rLm vaccines; challenged 3 or 4 weeks later by exposure to an aerosol generated by a nebulizer from a 10-ml single-cell suspension of Mtb Erdman strain (2.4×10⁴ CFU/ml) for 30 min followed by settling for 5 min; euthanized at 10 weeks post challenge; and spleens and right lungs removed and assayed for bacillus burden as described by us previously²². Control mice were sham vaccinated i.d. with PBS or immunized i.d. or i.n. with 1×10⁶ CFU BCG at Week 0.

Immunization of Guinea Pigs and Lymphocyte Proliferation Assay of their Spleen and Lung Cells

Guinea pigs (male Hartley), 4/group, were immunized i.d. at Weeks 0, 3, and 6 with 10⁶ CFU of the LmVector or rLm9Ag, bled and euthanized 6 days post the last immunization. Spleens and lungs were removed and single cell suspensions of spleen and lung cells prepared and stimulated with or without Mtb antigens. Lymphocyte proliferation using Flow cytometry analysis was assayed as described below.

Briefly, single cell suspensions of 1×10⁷ spleen and lung cells were stained with 1 μM Cell Tracer Violet (CTV, ThermoFisher, labeling cells to trace multiple generations using dye dilution by flow cytometry) for 10 min at 37° C. and washed with Phosphate buffer saline (PBS) supplemented with 5% fetal bovine serum. CTV treated cells were resuspended in T cell medium 22, adjusted to 5×10⁷ cells/ml, seeded in 96-well round-bottom plates (NUNC) (5×10⁶ cells per 0.1 ml per well) and incubated with or without 15-mer peptide pools of each of the 9 Mtb antigens (Ag85B, EspA, EsxA, EsxB, EsxH, EsxN, Mpt64, PPE68, TB8.4) (1 μg/ml per peptide) (PepMix, JPT Peptide Technologies, Berlin, Germany) or PPD (5 μg/ml). Cells incubated with T-cell medium alone served as a negative control and cells incubated with ConA (5 μg/ml) served as a positive control. After 4-days incubation at 37° C. in a CO2 incubator, cells were collected, washed with PBS, and stained with LIVE/DEAD Fixable Near-IR Dead Cell (LD-NIR) (ThermoFisher), followed by staining with cell surface markers of PanT-APC (BioRad), CD4-PE (BioRad), and CD8-FITC (BioRad). Lymphocyte proliferation was analyzed as loss of CTV staining (CTVLow) using an AttuneNxt flow cytometer (ThermoFisher). Data were analyzed using FlowJo software. Initial gating of the events included lymphocytes based on forward scatter vs. side scatter pattern, followed by selection for singlet cells, live PanT+ cells, and subsequently CD4+ and CD8+ T cells. Proliferating live CD4+ and CD8+ T cells were identified by loss of CTV staining (CTVLow) on each of the cell populations. The gates for each cell population were determined by using the cells incubated without addition of antigen and verified by cells incubated with addition of ConA.

Immunization and aerosol challenge of guinea pigs with virulent Mtb Erdman strain.

Groups of guinea pigs (Hartley, male), 10/group, were vaccinated i.d. once with 10³ CFU BCG (positive control), or s.q. three times at Weeks 0, 3, and 6 with one dose (10⁶) of LmVector (negative control), or 3 escalating doses (10⁵, 10⁶, and 10⁷) of rLm9Ag; challenged at Week 10 by aerosol with Mtb Erdman strain (2.4×10⁴ CFU/ml); euthanized at Week 20; and spleens and right lungs removed and assayed for bacillus burden as described by us previously 22.

Vaccine Stability after Passaging in Guinea Pigs

Guinea pigs were immunized subcutaneously at the back of the neck area with 1×10⁶ rLm9Ag vaccine diluted in 0.1 ml PBS. At 0, 1, 2, 4, and 8 days post immunization, 2 guinea pigs were euthanized at each time point; the skin at the immunization site (˜1 cm²), spleen, lung and liver of each animal were removed and homogenized in phosphate buffered saline (PBS); and the homogenates were serially diluted in PBS and plated onto BHI agar plates supplemented with streptomycin (200 μg/ml). The plates were incubated for 2 days at 37° C. in a CO₂ incubator. Bacterial colonies were recovered from plates of the various tissue homogenates at 0, 1, 2, and 4 days, but not at 8 days post immunization. Recovered colonies were randomly selected and inoculated into 1 ml BHI broth plus streptomycin (200 μg/ml) and grown overnight without agitation. The bacteria were then collected from the overnight culture, lysed in SDS buffer, and subjected to SDS-PAGE and western blotting using a rabbit polyclonal antibody to Lm ActA (AK18).

### Statistics and Reproducibility

Two-way ANOVA with Sidak's multiple comparisons test was performed to determine significance in comparisons of mean frequencies of cytokine-producing CD4+ and CD8+ T cells (FIGS. 2, 3, 4 and 5 of Jia et al.); one-way ANOVA with Tukey's multiple comparisons test was used to determine mean organ CFU among mice in vaccinated and control groups (FIGS. 6b, 7, 8 of Jia et al.). Two-way ANOVA with Sidak's multiple comparisons test was performed to determine significance in comparisons of mean frequencies of lymphocytes, CD4+, and CD8+ T cells between guinea pigs vaccinated with LmVector and those vaccinated with rLm9Ag (FIG. 9 of Jia et al.). Two-way ANOVA with Tukey's multiple comparisons test was used to determine significance in comparisons of percent weight change between guinea pigs vaccinated with LmVector and those vaccinated with rLm9Ag (FIG. 10b of Jia et al.) and one-way ANOVA with Tukey's multiple comparisons test was used to determine significance in comparison of mean organ CFU among guinea pigs in vaccinated and control groups (FIG. 10c of Jia et al.). All the statistical analyses were performed using Prism (9.2.0) software (GraphPad, San Diego, CA) except the Shapiro-Wilks tests. The Shapiro-Wilks test confirmed that all of the log scale CFU data in efficacy studies shown in FIGS. 7, 8 and 10 of Jia et al. have a normal distribution. Immunogenicity and efficacy studies were carried out in three animal models. The guinea pig immunogenicity study was repeated once.

### Example 2 References

1 Bloom, B. R. New Promise for Vaccines against Tuberculosis. N Engl J Med 379, 1672-1674 (2018). https://doi.org: 10.1056/NEJMe1812483
2 WHO. Global Tuberculosis Report 2021, <https://www.who.int/publications/i/item/9789240037021> (2021).
3 Knight, G. M. et al. Impact and cost-effectiveness of new tuberculosis vaccines in low- and middle-income countries. Proc Natl Acad Sci USA 111, 15520-15525 (2014). https://doi.org: 10.1073/pnas. 1404386111
4 Horwitz, M. A., Harth, G., Dillon, B. J. & Maslesa-Galic, S. Recombinant bacillus calmette-guerin (BCG) vaccines expressing the Mycobacterium tuberculosis 30-kDa major secretory protein induce greater protective immunity against tuberculosis than conventional BCG vaccines in a highly susceptible animal model. Proc Natl Acad Sci USA 97, 13853-13858 (2000). https://doi.org: 10.1073/pnas.250480397
5 Horwitz, M. A. & Harth, G. A new vaccine against tuberculosis affords greater survival after challenge than the current vaccine in the guinea pig model of pulmonary tuberculosis. Infect Immun 71, 1672-1679 (2003). https://doi.org: 10.1128/IAI.71.4.1672-1679.2003
6 Horwitz, M. A., Harth, G., Dillon, B. J. & Maslesa-Galic, S. Extraordinarily few organisms of a live recombinant BCG vaccine against tuberculosis induce maximal cell-mediated and protective immunity. Vaccine 24, 443-451 (2006).
7 Hoft, D. F. et al. A new recombinant bacille Calmette-Guerin vaccine safely induces significantly enhanced tuberculosis-specific immunity in human volunteers. J Infect Dis 198, 1491-1501 (2008). https://doi.org: 10.1086/592450
8 Tullius, M. V., Harth, G., Maslesa-Galic, S., Dillon, B. J. & Horwitz, M. A. A Replication-Limited Recombinant Mycobacterium bovis BCG vaccine against tuberculosis designed for human immunodeficiency virus-positive persons is safer and more efficacious than BCG. Infect Immun 76, 5200-5214 (2008). https://doi.org: 10.1128/IAI.00434-08
9 Spertini, F. et al. Safety of human immunisation with a live-attenuated Mycobacterium tuberculosis vaccine: a randomised, double-blind, controlled phase I trial. Lancet Respir Med 3, 953-962 (2015). https://doi.org: 10.1016/S2213-2600 (15) 00435-X
10 Grode, L. et al. Safety and immunogenicity of the recombinant BCG vaccine VPM1002 in a phase 1 open-label randomized clinical trial. Vaccine 31, 1340-1348 (2013). https://doi.org: 10.1016/j.vaccine.2012.12.053
11 Dietrich, J. et al. Mucosal administration of Ag85B-ESAT-6 protects against infection with Mycobacterium tuberculosis and boosts prior bacillus Calmette-Guerin immunity. J Immunol 177, 6353-6360 (2006). https://doi.org: 10.4049/jimmunol. 177.9.6353
12 Mearns, H. et al. H1: IC31 vaccination is safe and induces long-lived TNF-alpha(+) IL-2 (+)CD4 T cell responses in M. tuberculosis infected and uninfected adolescents: A randomized trial. Vaccine 35, 132-141 (2017). https://doi.org: 10.1016/j.vaccine.2016.11.023
13 Billeskov, R., Elvang, T. T., Andersen, P. L. & Dietrich, J. The Hy Vac4 subunit vaccine efficiently boosts BCG-primed anti-mycobacterial protective immunity. PLOS One 7, e39909 (2012). https://doi.org: 10.1371/journal.pone.0039909
14 Norrby, M. et al. Safety and immunogenicity of the novel H4: IC31 tuberculosis vaccine candidate in BCG-vaccinated adults: Two phase I dose escalation trials. Vaccine 35, 1652-1661 (2017). https://doi.org: 10.1016/j.vaccine.2017.01.055
15 Skeiky, Y. A. et al. Differential immune responses and protective efficacy induced by components of a tuberculosis polyprotein vaccine, Mtb72F, delivered as naked DNA or recombinant protein. J Immunol 172, 7618-7628 (2004). https://doi.org: 10.4049/jimmunol. 172.12.7618
16 Van Der Meeren, O. et al. Phase 2b Controlled Trial of M72/AS01E Vaccine to Prevent Tuberculosis. N Engl J Med 379, 1621-1634 (2018). https://doi.org: 10.1056/NEJMoal 803484
17 Coler, R. N. et al. The TLR-4 agonist adjuvant, GLA-SE, improves magnitude and quality of immune responses elicited by the ID93 tuberculosis vaccine: first-in-human trial. NPJ Vaccines 3, 34 (2018). https://doi.org: 10.1038/s41541-018-0057-5
18 Bertholet, S. et al. A defined tuberculosis vaccine candidate boosts BCG and protects against multidrug-resistant Mycobacterium tuberculosis. Sci Transl Med 2, 53ra74 (2010). https://doi.org: 10.1126/scitranslmed.3001094
19 Nemes, E. et al. Safety and Immunogenicity of Newborn MVA85A Vaccination and Selective, Delayed Bacille Calmette-Guerin for Infants of Human Immunodeficiency Virus-Infected Mothers: A Phase 2 Randomized, Controlled Trial. Clin Infect Dis 66, 554-563 (2018). https://doi.org: 10.1093/cid/cix834
20 Goonetilleke, N. P. et al. Enhanced immunogenicity and protective efficacy against Mycobacterium tuberculosis of bacille Calmette-Guerin vaccine using mucosal administration and boosting with a recombinant modified vaccinia virus Ankara. J Immunol 171, 1602-1609 (2003). https://doi.org: 10.4049/jimmunol. 171.3.1602
21 Juarez-Rodriguez, M. D. et al. Live attenuated Salmonella vaccines displaying regulated delayed lysis and delayed antigen synthesis to confer protection against Mycobacterium tuberculosis. Infect Immun 80, 815-831 (2012). https://doi.org: 10.1128/IAI.05526-11
22 Jia, Q., Dillon, B. J., Maslesa-Galic, S. & Horwitz, M. A. Listeria-vectored vaccine expressing the Mycobacterium tuberculosis 30-kilodalton major secretory protein via the constitutively active prfA* regulon boosts Mycobacterium bovis BCG efficacy against tuberculosis. Infect Immun 85 (2017). https://doi.org: 10.1128/IAI.00245-17
23 Jia, Q., Maslesa-Galic, S., Nava, S. & Horwitz, M. A. Listeria-vectored multiantigenic tuberculosis vaccine enhances protective immunity against aerosol challenge with virulent Mycobacterium tuberculosis in BCG-immunized C57BL/6 and BALB/c mice.mBio 13, e0068722 (2022). https://doi.org: 10.1128/mbio.00687-22
24 Chavez-Arroyo, A. & Portnoy, D. A. Why is Listeria monocytogenes such a potent inducer of CD8+ T-cells? Cell Microbiol 22, e13175 (2020). https://doi.org: 10.1111/cmi. 13175
25 Lauer, P. et al. Constitutive Activation of the PrfA regulon enhances the potency of vaccines based on live-attenuated and killed but metabolically active Listeria monocytogenes strains. Infect Immun 76, 3742-3753 (2008). https://doi.org: 10.1128/IAI.00390-08
26 Brockstedt, D. G. et al. Listeria-based cancer vaccines that segregate immunogenicity from toxicity. Proc Natl Acad Sci USA 101, 13832-13837 (2004).
27 Yan, L. et al. Selected prfA* mutations in recombinant attenuated Listeria monocytogenes strains augment expression of foreign immunogens and enhance vaccine-elicited humoral and cellular immune responses. Infect Immun 76, 3439-3450 (2008). https://doi.org: 10.1128/IAI.00245-08
28 Qiu, J. et al. Intranasal vaccination with the recombinant Listeria monocytogenes DeltaactA prfA* mutant elicits robust systemic and pulmonary cellular responses and secretory mucosal IgA. Clin Vaccine Immunol 18, 640-646 (2011). https://doi.org: 10.1128/CVI.00254-10
29 Arko-Mensah, J. et al. Resistance to mycobacterial infection: a pattern of early immune responses leads to a better control of pulmonary infection in C57BL/6 compared with BALB/c mice. Vaccine 27, 7418-7427 (2009). https://doi.org: 10.1016/j.vaccine.2009.06.110
30 Wakeham, J., Wang, J. & Xing, Z. Genetically determined disparate innate and adaptive cell-mediated immune responses to pulmonary Mycobacterium bovis BCG infection in C57BL/6 and BALB/c mice. Infect Immun 68, 6946-6953 (2000). https://doi.org: 10.1128/IAI.68.12.6946-6953.2000
31 Lefford, M. J. in The Mycobacteria: a sourcebook (eds George P. Kubica & Lawrence G. Wayne) 947-977 (Dekker, 1984).
32 Lu, Y. J. et al. CD4 T cell help prevents CD8 T cell exhaustion and promotes control of Mycobacterium tuberculosis infection. Cell Rep 36, 109696 (2021). https://doi.org: 10.1016/j.celrep.2021.109696
33 Chen, C. Y. et al. A critical role for CD8 T cells in a nonhuman primate model of tuberculosis. PLOS Pathog 5, e1000392 (2009). https://doi.org: 10.1371/journal.ppat. 1000392
34 Le, D. T. et al. A live-attenuated Listeria vaccine (ANZ-100) and a live-attenuated Listeria vaccine expressing mesothelin (CRS-207) for advanced cancers: phase I studies of safety and immune induction. Clin Cancer Res 18, 858-868 (2012). https://doi.org: 10.1158/1078-0432.CCR-11-2121
35 Whitney, J. B. et al. Prior exposure to an attenuated Listeria vaccine does not reduce immunogenicity: pre-clinical assessment of the efficacy of a Listeria vaccine in the induction of immune responses against HIV. J Immune Based Ther Vaccines 9, 2 (2011). https://doi.org: 10.1186/1476-8518-9-2
36 Leong, M. L. et al. Impact of preexisting vector-specific immunity on vaccine potency: characterization of Listeria monocytogenes-specific humoral and cellular immunity in humans and modeling studies using recombinant vaccines in mice. Infect Immun 77, 3958-3968 (2009). https://doi.org: 10.1128/IAI.01274-08
37 Lauer, P., Chow, M. Y., Loessner, M. J., Portnoy, D. A. & Calendar, R. Construction, characterization, and use of two Listeria monocytogenes site-specific phage integration vectors. J Bacteriol 184, 4177-4186 (2002).

### Polypeptide Sequences of MTB 5AG and 9AG Expression Cassettes

Note: In certain embodiments of the invention, the 9Ag fusion proteins are expressed from two fusion protein expression cassettes, 5Ag (ActAN-Mpt64-EsxH-EsxA-EsxB-Ag85B) and 5AgII (ActAN-Mpt64-EsxN-PPE68-EspA-TB8.4). The expression cassette for the 5Ag is integrated at the comK locus and the 5AgII is integrated at the tRNA^{arg} locus; 5Ag and 5AgII have a common antigen of Mpt64 as a leader protein. In both 5Ag and 5AgII fusion proteins, the Listeria monocytogenes ActAN (100 aa) is expressed as a N-terminus fusion to the Mtb proteins.

### Further Notes:

1) 5-antigen vaccine (rLm5Ag) expressing a fusion protein comprising Mycobacterium tuberculosis proteins 23.5/Mpt64, TB10.4/EsxH, ESAT6/EsxA, CFP10/EsxB, and r30/Antigen 85B; and
2) 9-antigen vaccine (rLm9Ag) expressing one fusion protein comprising Mycobacterium tuberculosis proteins 23.5/Mpt64, TB10.4/EsxH, ESAT6/EsxA, CFP10/EsxB, and r30/Antigen 85B at one locus of the Listeria monocytogenes chromosome and additionally expressing a second fusion protein comprising M. tuberculosis Mpt64-EsxN-PPE68-EspA-TB8.4 at a second locus of the Listeria monocytogenes chromosome. Since MPT64 is in both cassettes, the vaccine expresses 9 recombinant M. tuberculosis proteins.

The vaccines are intended as 1) booster vaccines for hosts initially immunized with BCG or another mycobacterial vaccine or 2) standalone vaccines in hosts not previously immunized with BCG or another mycobacterial vaccine.

Note: The vector for the vaccine, Listeria monocytogenes ΔactA ΔinlB prfA*, was originally provided by Aduro BioTech (evidently now merged into Chinook Therapeutics).

ActAN (100 aa)
(SEQ ID NO: 5)
VGLNRFMRAMMVVFITANCITINPDIIFAATDSEDSSLNTDEWEEEKTE
EQPSEVNTGPRYETAREVSSRDIEELEKSNKVKNTNKADLIAMLKAKAE
KG
5Ag (ActAN-Mpt64-EsxH-EsxA-EsxB-Ag85B)
(SEQ ID NO: 1)
VGLNRFMRAMMVVFITANCITINPDIIFAATDSEDSSLNTDEWEEEKTE
EQPSEVNTGPRYETAREVSSRDIEELEKSNKVKNTNKADLIAMLKAKAE
KGGSMAPKTYCEELKGTDTGQACQIQMSDPAYNINISLPSYYPDQKSLE
NYIAQTRDKFLSAATSSTPREAPYELNITSATYQSAIPPRGTQAVVLKV
YQNAGGTHPTTTYKAFDWDQAYRKPITYDTLWQADTDPLPVVFPIVQGE
LSKQTGQQVSIAPNAGLDPVNYQNFAVTNDGVIFFFNPGELLPEAAGPT
QVLVPRSAIDSMLARPMSQIMYNYPAMLGHAGDMAGYAGTLQSLGAEIA
VEQAALQSAWQGDTGITYQAWQAQWNQAMEDLVRAYHAMSSTHEANTMA
MMARDTAEAAKWGGGGSGMTEQQWNFAGIEAAASAIQGNVTSIHSLLDE
GKQSLTKLAAAWGGSGSEAYQGVQQKWDATATELNNALQNLARTISEAG
QAMASTEGNVTGMFAGSSGGSSGMAEMKTDAATLAQEAGNFERISGDLK
TQIDQVESTAGSLQGQWRGAAGTAAQAAVVRFQEAANKQKQELDEISTN
IRQAGVQYSRADEEQQQALSSQMGFGSSGGSSGAFSRPGLPVEYLQVPS
PSMGRDIKVQFQSGGNNSPAVYLLDGLRAQDDYNGWDINTPAFEWYYQS
GLSIVMPVGGQSSFYSDWYSPACGKAGCQTYKWETFLTSELPQWLSANR
AVKPTGSAAIGLSMAGSSAMILAAYHPQQFIYAGSLSALLDPSQGMGPS
LIGLAMGDAGGYKAADMWGPSSDPAWERNDPTQQIPKLVANNTRLWVYC
GNGTPNELGGANIPAEFLENFVRSSNLKFQDAYNAAGGHNAVFNFPPNG
THSWEYWGAQLNAMKGDLQSSLGAG
Theoretical pI/Mw: 4.66/97360.01
9Ag
5Ag (ActAN-Mpt64-EsxH-EsxA-EsxB-Ag85B): the same
as above 5AgII (ActAN-Mpt64-EsxN-PPE68-EspA-TB8.4)
(SEQ ID NO: 2)
VGLNRFMRAMMVVFITANCITINPDIIFAATDSEDSSLNTDEWEEEKTEE
QPSEVNTGPRYETAREVSSRDIEELEKSNKVKNTNKADLIAMLKAKAEKG
GSMAPKTYCEELKGTDTGQACQIQMSDPAYNINISLPSYYPDQKSLENYI
AQTRDKFLSAATSSTPREAPYELNITSATYQSAIPPRGTQAVVLKVYQNA
GGTHPTTTYKAFDWDQAYRKPITYDTLWQADTDPLPVVFPIVQGELSKQT
GQQVSIAPNAGLDPVNYQNFAVTNDGVIFFFNPGELLPEAAGPTQVLVPR
SAIDSMLAMTINYQFGDVDAHGAMIRAQAASLEAEHQAIVRDVLAAGDFW
GGAGSVACQEFITQLGRNFQVIYEQANAHGQKVQAAGNNMAQTDSAVGSS
WAGGSGMLWHAMPPELNTARLMAGAGPAPMLAAAAGWQTLSAALDAQAVE
LTARLNSLGEAWTGGGSDKALAAATPMVVWLQTASTQAKTRAMQATAQAA
AYTQAMATTPSLPEIAANHITQAVLTATNFFGINTIPIALTEMDYFIRMW
NQAALAMEVYQAETAVNTLFEKLEPMASILDPGASQSTTNPIFGMPSPGS
STPVGQLPPAATQTLGQLGEMSGPMQQLTQPLQQVTSLFSQVGGTGGGNP
ADEEAAQMGLLGTSPLSNHPLAGGSGPSAGAGLLRAESLPGAGGSLTRTP
LMSQLIEKPVAPSVMPAAAAGSSATGGAAPVGAGAMGQGAQSGGSTRPGL
VAPAPLAQEREEDDEDDWDEEDDWGSSGGSSGAMSRAFIIDPTISAIDGL
YDLLGIGIPNQGGILYSSLEYFEKALEELAAAFPGDGWLGSAADKYAGKN
RNHVNFFQELADLDRQLISLIHDQANAVQTTRDILEGAKKGLEGEVWEFI
TNALNGLKELWDKLTGWVTGLFSRGWSNLESFFAGVPGLTGATSGLSQVT
GLFGAAGLSASSGLAHADSLASSASLPALAGIGGGSGFGGLPSLAQVHAA
STRQALRPRADGPVGAAAEQVGGQSQLVSAQGSQGMGGPVGMGGMHPSSG
ASKGTTTKKYSEGAAAGTEDAERAPVEADAGGGQKVLVRNVVGSSGGSSG
AMDPVDAVINTTCNYGQVVAALNATDPGAAAQFNASPVAQSYLRNFLAA
PPPQRAAMAAQLQAVPGAAQYIGLVESVAGSCNNY
Theoretical pI/Mw: 4.59/122612.90

M. tuberculosis polynucleotide and polypeptide sequences are well known in the art as a result of the M. tuberculosis genome project. See, e.g. UniProt Designation “P9WIN9” for Mycobacterium tuberculosis Immunogenic protein MPT64 (“23.5/Mpt64”). See, e.g. UniProt Designation “P9WNK3” for Mycobacterium tuberculosis ESAT-6-like protein EsxH (“TB10.4/EsxH”). See, e.g. UniProt Designation “P9WNK7” for Mycobacterium tuberculosis 6 kDa early secretory antigenic target (“ESAT6/EsxA”). See, e.g. UniProt Designation “P9WNK5” for Mycobacterium tuberculosis ESAT-6-like protein EsxB (“CFP10/EsxB”). See, e.g. UniProt Designation “P9WQP1” for Mycobacterium tuberculosis Diacylglycerol acyltransferase/mycolyltransferase Ag85B (“r30/Antigen 85B”). See, e.g. UniProt Designation “P9WNJ3” for Mycobacterium tuberculosis ESAT-6-like protein EsxN (“EsxN”). See, e.g. UniProt Designation “P9WHW9” for Mycobacterium tuberculosis PPE family immunomodulator PPE68 (“PPE68”). See, e.g. UniProt Designation “P9WJE1” for Mycobacterium tuberculosis ESX-1 secretion-associated protein EspA (“EspA”). See, e.g. UniProt Designation “O50430” for Mycobacterium tuberculosis Low molecular weight T-cell antigen TB8.4 (“TB8.4”).

See also: Cole et al. Nature. Nature 393:537-544, 1998. See also U.S. Pat. Nos. 7,622,107; 7,300,660; 7,002,002; 6,924,118; 6,818,223; 6,761,894; 6,752,993; 6,599,510; 6,471,967; 6,054,133; 6,013,660; and 5,108,745; and U.S. patents application Nos. 20110129492; 20100284963; 20100183547; and 20100092518, the contents of which are incorporated by reference. M. tuberculosis Protein and coding sequences can also be found for example by an online search using the terms: genolist.pasteur.fr/TubercuList/.

It is intended that the scope of the invention be limited not by this detailed description, but rather by the claims appended hereto. The above specification, examples and data provide a complete description of the manufacture and use of the apparatus and method of the invention. Since many embodiments of the invention can be made without departing from the scope of the invention, the invention resides in the claims hereinafter appended and the equivalents thereto.

References, the contents of which are incorporated by reference.
1. Lange C, Aaby P, Behr M A, Donald P R, Kaufmann SHE, Netea M G, Mandalakas A M. 2022. 100 years of Mycobacterium bovis bacille Calmette-Guerin. Lancet Infect Dis 22: e2-e12.
2. Chard A N, Gacic-Dobo M, Diallo M S, Sodha S V, Wallace A S. 2020. Routine Vaccination Coverage-Worldwide, 2019. MMWR Morb Mortal Wkly Rep 69:1706-1710.
3. Horwitz M A, Harth G, Dillon B J, Maslesa-Galic S. 2000. Recombinant bacillus calmette-guerin (BCG) vaccines expressing the Mycobacterium tuberculosis 30-kDa major secretory protein induce greater protective immunity against tuberculosis than conventional BCG vaccines in a highly susceptible animal model. Proc Natl Acad Sci USA 97:13853-13858.
4. Hoft D F, Blazevic A, Abate G, Hanekom W A, Kaplan G, Soler J H, Weichold F, Geiter L, Sadoff J C, Horwitz M A. 2008. A new recombinant bacille Calmette-Guerin vaccine safely induces significantly enhanced tuberculosis-specific immunity in human volunteers. J Infect Dis 198:1491-1501.
5. Sable S B, Posey J E, Scriba T J. 2019. Tuberculosis Vaccine Development: Progress in Clinical Evaluation. Clin Microbiol Rev 33.
6. Fatima S, Kumari A, Das G, Dwivedi V P. 2020. Tuberculosis vaccine: A journey from BCG to present. Life Sci 252:117594.
7. van Zyl-Smit R N, Esmail A, Bateman M E, Dawson R, Goldin J, van Rikxoort E, Douoguih M, Pau M G, Sadoff J C, McClain J B, Snowden M A, Benko J, Hokey D A, Rutkowski K T, Graves A, Shepherd B, Ishmukhamedov S, Kagina BMN, Abel B, Hanekom W A, Scriba T J, Bateman E D. 2017. Safety and Immunogenicity of Adenovirus 35 Tuberculosis Vaccine Candidate in Adults with Active or Previous Tuberculosis. A Randomized Trial. Am J Respir Crit Care Med 195:1171-1180.
8. Nemes E, Geldenhuys H, Rozot V, Rutkowski K T, Ratangee F, Bilek N, Mabwe S, Makhethe L, Erasmus M, Toefy A, Mulenga H, Hanekom W A, Self S G, Bekker L G, Ryall R, Gurunathan S, DiazGranados C A, Andersen P, Kromann I, Evans T, Ellis R D, Landry B, Hokey D A, Hopkins R, Ginsberg A M, Scriba T J, Hatherill M, Team C S. 2018. Prevention of M. tuberculosis Infection with H4: IC31 Vaccine or BCG Revaccination. N Engl J Med 379:138-149.
9. Hansen S G, Zak D E, Xu G, Ford J C, Marshall E E, Malouli D, Gilbride R M, Hughes C M, Ventura A B, Ainslie E, Randall K T, Selseth A N, Rundstrom P, Herlache L, Lewis M S, Park H, Planer S L, Turner J M, Fischer M, Armstrong C, Zweig R C, Valvo J, Braun J M, Shankar S, Lu L, Sylwester A W, Legasse A W, Messerle M, Jarvis M A, Amon L M, Aderem A, Alter G, Laddy D J, Stone M, Bonavia A, Evans T G, Axthelm M K, Fruh K, Edlefsen P T, Picker L J. 2018. Prevention of tuberculosis in rhesus macaques by a cytomegalovirus-based vaccine. Nat Med 24:130-143.
10. Lauer P, Hanson B, Lemmens E E, Liu W, Luckett W S, Leong M L, Allen H E, Skoble J, Bahjat K S, Freitag N E, Brockstedt D G, Dubensky T W, Jr. 2008. Constitutive Activation of the PrfA regulon enhances the potency of vaccines based on live-attenuated and killed but metabolically active Listeria monocytogenes strains. Infect Immun 76:3742-3753.
11. Portnoy D A, Auerbuch V, Glomski I J. 2002. The cell biology of Listeria monocytogenes infection: the intersection of bacterial pathogenesis and cell-mediated immunity. J Cell Biol 158:409-414.
12. Whitney J B, Mirshahidi S, Lim S Y, Goins L, Ibegbu C C, Anderson D C, Raybourne R B, Frankel F R, Lieberman J, Ruprecht R M. 2011. Prior exposure to an attenuated Listeria vaccine does not reduce immunogenicity: pre-clinical assessment of the efficacy of a Listeria vaccine in the induction of immune responses against HIV. J Immune Based Ther Vaccines 9:2.
13. Starks H, Bruhn K W, Shen H, Barry R A, Dubensky T W, Brockstedt D, Hinrichs D J, Higgins D E, Miller J F, Giedlin M, Bouwer FIG. 2004. Listeria monocytogenes as a vaccine vector: virulence attenuation or existing antivector immunity does not diminish therapeutic efficacy. J Immunol 173:420-427.
14. Le D T, Brockstedt D G, Nir-Paz R, Hampl J, Mathur S, Nemunaitis J, Sterman D H, Hassan R, Lutz E, Moyer B, Giedlin M, Louis J L, Sugar E A, Pons A, Cox A L, Levine J, Murphy A L, Illei P, Dubensky T W, Jr., Eiden J E, Jaffee E M, Laheru D A. 2012. A live-attenuated Listeria vaccine (ANZ-100) and a live-attenuated Listeria vaccine expressing mesothelin (CRS-207) for advanced cancers: phase I studies of safety and immune induction. Clin Cancer Res 18:858-868.
15. Jia Q, Dillon B J, Maslesa-Galic S, Horwitz M A. 2017. Listeria-Vectored Vaccine Expressing the Mycobacterium tuberculosis 30-Kilodalton Major Secretory Protein via the Constitutively Active prfA* Regulon Boosts Mycobacterium bovis BCG Efficacy against Tuberculosis. Infect Immun 85.
16. Lauer P, Chow M Y, Loessner M J, Portnoy D A, Calendar R. 2002. Construction, characterization, and use of two Listeria monocytogenes site-specific phage integration vectors. J Bacteriol 184:4177-4186.
17. Jia Q, Bowen R, Lee B Y, Dillon B J, Maslesa-Galic S, Horwitz M A. 2016. Francisella tularensis Live Vaccine Strain deficient in capB and overexpressing the fusion protein of IglA, IglB, and IgIC from the bfr promoter induces improved protection against F. tularensis respiratory challenge. Vaccine 34:4969-4978.
18. Kapopoulou A, Lew J M, Cole S T. 2011. The MycoBrowser portal: A comprehensive and manually annotated resource for mycobacterial genomes. Tuberculosis 91:8-13.
19. Hervas-Stubbs S, Majlessi L, Simsova M, Morova J, Rojas M J, Nouze C, Brodin P, Sebo P, Leclerc C. 2006. High frequency of CD4+ T cells specific for the TB10.4 protein correlates with protection against Mycobacterium tuberculosis infection. Infect Immun 74:3396-3407.
20. Coler R N, Campos-Neto A, Ovendale P, Day F H, Fling S P, Zhu L, Serbina N, Flynn J L, Reed S G, Alderson M R. 2001. Vaccination with the T cell antigen Mtb 8.4 protects against challenge with Mycobacterium tuberculosis. J Immunol 166:6227-6235.
21. Nandakumar S, Kannanganat S, Dobos K M, Lucas M, Spencer J S, Amara R R, Plikaytis B B, Posey J E, Sable S B. 2016. Boosting BCG-primed responses with a subunit Apa vaccine during the waning phase improves immunity and imparts protection against Mycobacterium tuberculosis. Sci Rep 6:25837.
22. Horwitz M A, Lee BWE, Dillon B J, Harth G. 1995. Protective Immunity against Tuberculosis Induced by Vaccination with Major Extracellular Proteins of Mycobacterium-Tuberculosis. Proceedings of the National Academy of Sciences of the United States of America 92:1530-1534.
23. Horwitz M A, Harth G. 2003. A new vaccine against tuberculosis affords greater survival after challenge than the current vaccine in the guinea pig model of pulmonary tuberculosis. Infect Immun 71:1672-1679.
24. Luo X D, Zhu D Y, Chen Q, Jiang Y, Jiang S, Yang C. 2004. [A study of the protective effect of the DNA vaccine encoding tubercle antigen 85B with MPT64 in mice challenged with Mycobacterium tuberculosis]. Zhonghua Jie He He Hu Xi Za Zhi 27:611-616.
25. Sali M, Di Sante G, Cascioferro A, Zumbo A, Nicolo C, Dona V, Rocca S, Procoli A, Morandi M, Ria F, Palu G, Fadda G, Manganelli R, Delogu G. 2010. Surface expression of MPT64 as a fusion with the P E domain of P E PGRS33 enhances Mycobacterium bovis BCG protective activity against Mycobacterium tuberculosis in mice. Infect Immun 78:5202-5213.
26. Dey B, Jain R, Gupta U D, Katoch V M, Ramanathan V D, Tyagi A K. 2011. A booster vaccine expressing a latency-associated antigen augments BCG induced immunity and confers enhanced protection against tuberculosis. PLOS One 6: e23360.
27. Shi C, Chen L, Chen Z, Zhang Y, Zhou Z, Lu J, Fu R, Wang C, Fang Z, Fan X. 2010. Enhanced protection against tuberculosis by vaccination with recombinant BCG over-expressing HspX protein. Vaccine 28:5237-5244.
28. Ma J, Tian M, Fan X, Yu Q, Jing Y, Wang W, Li L, Zhou Z. 2016. Mycobacterium tuberculosis multistage antigens confer comprehensive protection against pre- and post-exposure infections by driving Th1-type T cell immunity. Oncotarget 7:63804-63815.
29. Mollenkopf H J, Grode L, Mattow J, Stein M, Mann P, Knapp B, Ulmer J, Kaufmann S H. 2004. Application of mycobacterial proteomics to vaccine design: improved protection by Mycobacterium bovis BCG prime-Rv3407 DNA boost vaccination against tuberculosis. Infect Immun 72:6471-6479.
30. Woodworth J S, Clemmensen H S, Battey H, Dijkman K, Lindenstrom T, Laureano R S, Taplitz R, Morgan J, Aagaard C, Rosenkrands I, Lindestam Arlehamn C S, Andersen P, Mortensen R. 2021. A Mycobacterium tuberculosis-specific subunit vaccine that provides synergistic immunity upon co-administration with Bacillus Calmette-Guerin. Nat Commun 12:6658.
31. Wu Y, Woodworth J S, Shin D S, Morris S, Behar S M. 2008. Vaccine-elicited 10-kilodalton culture filtrate protein-specific CD8+ T cells are sufficient to mediate protection against Mycobacterium tuberculosis infection. Infect Immun 76:2249-2255.
32. Weinrich Olsen A, van Pinxteren L A, Meng Okkels L, Birk Rasmussen P, Andersen P. 2001. Protection of mice with a tuberculosis subunit vaccine based on a fusion protein of antigen 85b and esat-6. Infect Immun 69:2773-2778.
33. Hoang T, Aagaard C, Dietrich J, Cassidy J P, Dolganov G, Schoolnik G K, Lundberg C V, Agger E M, Andersen P. 2013. ESAT-6 (EsxA) and TB10.4 (EsxH) based vaccines for pre- and post-exposure tuberculosis vaccination. PLOS One 8: e80579.
34. Tundup S, Pathak N, Ramanadham M, Mukhopadhyay S, Murthy K J, Ehtesham N Z, Hasnain S E. 2008. The co-operonic PE25/PPE41 protein complex of Mycobacterium tuberculosis elicits increased humoral and cell mediated immune response. PLOS One 3: e3586.
35. Demangel C, Brodin P, Cockle P J, Brosch R, Majlessi L, Leclerc C, Cole S T. 2004. Cell envelope protein PPE68 contributes to Mycobacterium tuberculosis RD1 immunogenicity independently of a 10-kilodalton culture filtrate protein and ESAT-6.
Infect Immun 72:2170-2176.
36. Behr M A. 2002. BCG—different strains, different vaccines? Lancet Infect Dis 2:86-92.
37. Abdallah A M, Behr M A. 2017. Evolution and Strain Variation in BCG. Adv Exp Med Biol 1019:155-169.
38. Aagaard C, Hoang T T, Izzo A, Billeskov R, Troudt J, Arnett K, Keyser A, Elvang T, Andersen P, Dietrich J. 2009. Protection and polyfunctional T cells induced by Ag85B-TB10.4/IC31 against Mycobacterium tuberculosis is highly dependent on the antigen dose. PLOS One 4: e5930.
39. Elvang T, Christensen J P, Billeskov R, Thi Kim Thanh Hoang T, Holst P, Thomsen A R, Andersen P, Dietrich J. 2009. CD4 and CD8 T cell responses to the M. tuberculosis Ag85B-TB10.4 promoted by adjuvanted subunit, adenovector or heterologous prime boost vaccination. PLOS One 4: e5139.
40. Juarez-Rodriguez M D, Arteaga-Cortes L T, Kader R, Curtiss R, 3rd, Clark-Curtiss J E. 2012. Live attenuated Salmonella vaccines against Mycobacterium tuberculosis with antigen delivery via the type III secretion system. Infect Immun 80:798-814.
41. Blander S J, Horwitz M A. 1989. Vaccination with the major secretory protein of Legionella pneumophila induces cell-mediated and protective immunity in a guinea pig model of Legionnaires' disease. J Exp Med 169:691-705.
42. Horwitz M A. 1997. A new T B vaccine. The Immunologist 5 (1): 15-20.
43. Huygen K, Abramowicz D, Vandenbussche P, Jacobs F, De Bruyn J, Kentos A, Drowart A, Van Vooren J P, Goldman M. 1992. Spleen cell cytokine secretion in Mycobacterium bovis BCG-infected mice. Infect Immun 60:2880-2886.
44. Wakeham J, Wang J, Xing Z. 2000. Genetically determined disparate innate and adaptive cell-mediated immune responses to pulmonary Mycobacterium bovis BCG infection in C57B L/6 and BALB/c mice. Infect Immun 68:6946-6953.
45. Jung Y J, Ryan L, LaCourse R, North R J. 2009. Differences in the ability to generate type 1 T helper cells need not determine differences in the ability to resist Mycobacterium tuberculosis infection among mouse strains. J Infect Dis 199:1790-1796.
46. Muller I, Cobbold S P, Waldmann H, Kaufmann S H. 1987. Impaired resistance to Mycobacterium tuberculosis infection after selective in vivo depletion of L3T4+ and Lyt-2+ T cells. Infect Immun 55:2037-2041.
47. Flynn J L, Goldstein M M, Triebold K J, Koller B, Bloom B R. 1992. Major histocompatibility complex class I-restricted T cells are required for resistance to Mycobacterium tuberculosis infection. Proc Natl Acad Sci USA 89:12013-12017.
48. Behar S M, Dascher C C, Grusby M J, Wang C R, Brenner M B. 1999. Susceptibility of mice deficient in CDID or TAPI to infection with Mycobacterium tuberculosis. J Exp Med 189:1973-1980.
49. Mogues T, Goodrich M E, Ryan L, LaCourse R, North R J. 2001. The relative importance of T cell subsets in immunity and immunopathology of airborne Mycobacterium tuberculosis infection in mice. J Exp Med 193:271-280.
50. Tascon R E, Stavropoulos E, Lukacs K V, Colston M J. 1998. Protection against Mycobacterium tuberculosis infection by CD8+ T cells requires the production of gamma interferon. Infect Immun 66:830-834.
51. Chen C Y, Huang D, Wang R C, Shen L, Zeng G, Yao S, Shen Y, Halliday L, Fortman J, McAllister M, Estep J, Hunt R, Vasconcelos D, Du G, Porcelli S A, Larsen M H, Jacobs W R, Jr., Haynes B F, Letvin N L, Chen Z W. 2009. A critical role for CD8 T cells in a nonhuman primate model of tuberculosis. PLOS Pathog 5: e1000392.

Those skilled in the art will appreciate that the exemplary discussions of M. tuberculosis that are provided herein are in no way intended to limit the scope of the present invention to the treatment of M. tuberculosis. Similarly, the teachings herein are not limited in any way to the treatment of tubercular infections. On the contrary, this invention may be used to advantageously provide safe and effective vaccines and immunotherapeutic agents against the immunogenic determinants of any pathogenic agent expressing extracellular products and thereby inhibit the infectious transmission of those organisms.

This concludes the description of embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

Those of skill in this art understand that aspects of this technology can be adapted to form a wide variety of embodiments of the invention. All literature and other references are incorporated herein by reference (e.g. U.S. Pat. Nos. 10,010,595, 7,300,660 and 7,002,002, and PCT Publications WO 01/46473 and WO 02/094848). Literature describing methods and materials that relate to embodiments of the invention includes Brockstedt et al., (2004) Proc Natl Acad Sci USA 101 (38): 13832-7. PMID 15365184; Brockstedt et al., (2005) Nat Med 11 (8): 853-60. PMID 16041382; Colditz et al., (1994).” JAMA 271 (9): 698-702. PMID 8309034; Fine, P. E. (1989) Rev Infect Dis 11 Suppl 2: S353-9. PMID 2652252; Harth et al., (1997) Infect Immun 65 (6): 2321-8. PMID 9169770; Horwitz et al., (1995) Proc Natl Acad Sci USA 92 (5): 1530-4. PMID 7878014; Horwitz et al., (2000) Proc Natl Acad Sci USA 97 (25): 13853-8. PMID 11095745; Horwitz et al., (2005) Infect Immun 73 (8): 4676-83. PMID 16040980; Jia et al., (2009) Vaccine 27 (8): 1216-29. PMID 19126421; Lauer et al., (2008) Infect Immun 76 (8): 3742-53. PMID 18541651; Lee et al., (2006) Infect Immun 74 (7): 4002-13. PMID 16790773; McShane et al., (2004) Nat Med 10 (11): 1240-4. PMID 15502839; Santosuosso et al., (2006) Infect Immun 74 (8): 4634-43. PMID 16861651; Vordermeier et al., (2009) Infect Immun 77 (8): 3364-73. PMID 19487476; Williams et al., (2005) Infect Immun 73 (6): 3814-6. PMID 15908420; Xing et al., (2009) PLOS One 4 (6): e5856. PMID 19516906; and Yan et al., (2008) Infect Immun 76 (8): 3439-50. PMID 18474644.

1. A composition of matter comprising at least one fusion protein having antigenic epitopes present in at least five Mycobacterium tuberculosis proteins selected from: immunogenic protein MPT64 (“23.5/Mpt64”), ESAT-6-like protein EsxH (“TB10.4/EsxH”), 6 kDa early secretory antigenic target (“ESAT6/EsxA”), ESAT-6-like protein EsxB (“CFP10/EsxB”), and diacylglycerol acyltransferase/mycolyltransferase Ag85B (“r30/Antigen 85B”); ESAT-6-like protein EsxN (“EsxN”); PPE family immunomodulator PPE68 (“PPE68”); ESX-1 secretion-associated protein EspA (“EspA”) and low molecular weight T-cell antigen TB8.4 (“TB8.4”), wherein the composition comprises live attenuated Listeria monocytogenes expressing the at least one fusion protein.

2. The composition of claim 1, wherein when administered to mice as a vaccine, the composition elicits an immune response to Mycobacterium tuberculosis exposure in the mice characterized by an at least 10% reduction in Mycobacterium tuberculosis colony forming units in lungs of mice administered the vaccine as compared to lungs of control mice administered a control composition lacking antigenic epitopes present in Mycobacterium tuberculosis.

3. The composition of claim 1, wherein:
the fusion protein comprises antigenic epitopes present in the polypeptide sequence:
(SEQ ID NO: 1)
VGLNRFMRAMMVVFITANCITINPDIIFAATDSEDSSLNTDEWEEEKTE
EQPSEVNTGPRYETAREVSSRDIEELEKSNKVKNTNKADLIAMLKAKAE
KGGSMAPKTYCEELKGTDTGQACQIQMSDPAYNINISLPSYYPDQKSLE
NYIAQTRDKFLSAATSSTPREAPYELNITSATYQSAIPPRGTQAVVLKV
YQNAGGTHPTTTYKAFDWDQAYRKPITYDTLWQADTDPLPVVFPIVQGE
LSKQTGQQVSIAPNAGLDPVNYQNFAVTNDGVIFFFNPGELLPEAAGPT
QVLVPRSAIDSMLARPMSQIMYNYPAMLGHAGDMAGYAGTLQSLGAEIA
VEQAALQSAWQGDTGITYQAWQAQWNQAMEDLVRAYHAMSSTHEANTMA
MMARDTAEAAKWGGGGSGMTEQQWNFAGIEAAASAIQGNVTSIHSLLDE
GKQSLTKLAAAWGGSGSEAYQGVQQKWDATATELNNALQNLARTISEAG
QAMASTEGNVTGMFAGSSGGSSGMAEMKTDAATLAQEAGNFERISGDLK
TQIDQVESTAGSLQGQWRGAAGTAAQAAVVRFQEAANKQKQELDEISTN
IRQAGVQYSRADEEQQQALSSQMGFGSSGGSSGAFSRPGLPVEYLQVPS
PSMGRDIKVQFQSGGNNSPAVYLLDGLRAQDDYNGWDINTPAFEWYYQS
GLSIVMPVGGQSSFYSDWYSPACGKAGCQTYKWETFLTSELPQWLSANR
AVKPTGSAAIGLSMAGSSAMILAAYHPQQFIYAGSLSALLDPSQGMGPS
LIGLAMGDAGGYKAADMWGPSSDPAWERNDPTQQIPKLVANNTRLWVYC
GNGTPNELGGANIPAEFLENFVRSSNLKFQDAYNAAGGHNAVFNFPPNG
THSWEYWGAQLNAMKGDLQSSLGAG;
and/or
the fusion protein comprises antigenic epitopes present in the polypeptide sequence:
(SEQ ID NO: 2)
VGLNRFMRAMMVVFITANCITINPDIIFAATDSEDSSLNTDEWEEEKTE
EQPSEVNTGPRYETAREVSSRDIEELEKSNKVKNTNKADLIAMLKAKAE
KGGSMAPKTYCEELKGTDTGQACQIQMSDPAYNINISLPSYYPDQKSLE
NYIAQTRDKFLSAATSSTPREAPYELNITSATYQSAIPPRGTQAVVLKV
YQNAGGTHPTTTYKAFDWDQAYRKPITYDTLWQADTDPLPVVFPIVQGE
LSKQTGQQVSIAPNAGLDPVNYQNFAVTNDGVIFFFNPGELLPEAAGPT
QVLVPRSAIDSMLAMTINYQFGDVDAHGAMIRAQAASLEAEHQAIVRDV
LAAGDFWGGAGSVACQEFITQLGRNFQVIYEQANAHGQKVQAAGNNMAQ
TDSAVGSSWAGGSGMLWHAMPPELNTARLMAGAGPAPMLAAAAGWQTLS
AALDAQAVELTARLNSLGEAWTGGGSDKALAAATPMVVWLQTASTQAKT
RAMQATAQAAAYTQAMATTPSLPEIAANHITQAVLTATNFFGINTIPIA
LTEMDYFIRMWNQAALAMEVYQAETAVNTLFEKLEPMASILDPGASQST
TNPIFGMPSPGSSTPVGQLPPAATQTLGQLGEMSGPMQQLTQPLQQVTS
LFSQVGGTGGGNPADEEAAQMGLLGTSPLSNHPLAGGSGPSAGAGLLRA
ESLPGAGGSLTRTPLMSQLIEKPVAPSVMPAAAAGSSATGGAAPVGAGA
MGQGAQSGGSTRPGLVAPAPLAQEREEDDEDDWDEEDDWGSSGGSSGAM
SRAFIIDPTISAIDGLYDLLGIGIPNQGGILYSSLEYFEKALEELAAAF
PGDGWLGSAADKYAGKNRNHVNFFQELADLDRQLISLIHDQANAVQTTR
DILEGAKKGLEGEVWEFITNALNGLKELWDKLTGWVTGLFSRGWSNLES
FFAGVPGLTGATSGLSQVTGLFGAAGLSASSGLAHADSLASSASLPALA
GIGGGSGFGGLPSLAQVHAASTRQALRPRADGPVGAAAEQVGGQSQLVS
AQGSQGMGGPVGMGGMHPSSGASKGTTTKKYSEGAAAGTEDAERAPVEA
DAGGGQKVLVRNVVGSSGGSSGAMDPVDAVINTTCNYGQVVAALNATDP
GAAAQFNASPVAQSYLRNFLAAPPPQRAAMAAQLQAVPGAAQYIGLVES
VAGSCNNY.

4. The composition of claim 1, wherein the Listeria monocytogenes:
does not express a functional InlB protein;
does not express a functional actA protein; and/or
expresses prfA protein having a G155S substitution mutation.

5. The composition of claim 1, wherein the composition comprises at least two fusion proteins.

6. The composition of claim 5, wherein the Listeria monocytogenes expresses a first fusion protein encoded by a polynucleotide present in a first locus of the Listeria monocytogenes genome and a second fusion protein encoded by a polynucleotide present in a second locus of the Listeria monocytogenes genome.

7. The composition of claim 6, wherein the first fusion protein comprises epitopes present in at least two Mycobacterium tuberculosis proteins selected from: 23.5/Mpt64, TB10.4/EsxH, ESAT6/EsxA, CFP10/EsxB, and r30/Antigen 85B; and the second fusion protein comprises epitopes present in at least two Mycobacterium tuberculosis proteins selected from EsxN; PPE68; EspA and TB8.4.

8. The composition of claim 1, wherein immunogenic epitopes are disposed on the fusion protein such that immunogenic epitopes of 23.5/Mpt64 are N-terminal to other Mycobacterium tuberculosis immunogenic epitopes disposed in the fusion protein.

9. The composition of claim 1, wherein immunogenic epitopes are disposed on the fusion protein such that immunogenic epitopes of r30/Antigen 85B are C-terminal to other Mycobacterium tuberculosis immunogenic epitopes disposed in the fusion protein.

10. A method of generating an immune response to a Mycobacterium tuberculosis in a mammal comprising administering to the mammal a composition of claim 1 such that an immune response to Mycobacterium tuberculosis is generated.

11. The method of claim 10, further comprising immunizing the mammal with Mycobacterium bovis strain Bacille Calmette-Guérin (BCG).

12. The method of claim 11, wherein the BCG is used in a primary immunization and the Listeria monocytogenes expressing the at least one fusion protein is used in a booster immunization.

13. The method of claim 11, wherein the Listeria monocytogenes expressing the at least one fusion protein is used in a primary immunization and the BCG is used in a booster immunization.

14. The method of claim 10, wherein the mammal is immunized with the composition intranasally, subcutaneously, intradermally, intramuscularly or orally.

15. The method of claim 10, wherein the composition is administered subcutaneously.

16. The method of claim 10, wherein the composition is administered intradermally.

17. The method of claim 10, wherein the mammal is a human.

18. The method of claim 10, wherein the at least one fusion protein does not comprise immunogenic epitopes present in at least one Mycobacterium tuberculosis protein selected from Hypoxic response protein 1 (“Hrp1”), PE-PGRS family protein PE25 (“PE25”), Alpha-crystallin (“HSPX”) and Antitoxin VapB47 (“VapB47”).

19. The method of claim 10, wherein the at least one fusion protein comprises Mycobacterium tuberculosis immunogenic epitopes consisting essentially of immunogenic epitopes present in at least 5 Mycobacterium tuberculosis proteins selected from: immunogenic protein MPT64 (“23.5/Mpt64”), ESAT-6-like protein EsxH (“TB10.4/EsxH”), 6 kDa early secretory antigenic target (“ESAT6/EsxA”), ESAT-6-like protein EsxB (“CFP10/EsxB”), and diacylglycerol acyltransferase/mycolyltransferase Ag85B (“r30/Antigen 85B”); ESAT-6-like protein EsxN (“EsxN”); PPE family immunomodulator PPE68 (“PPE68”); ESX-1 secretion-associated protein EspA (“EspA”) and low molecular weight T-cell antigen TB8.4 (“TB8.4”).

20. Use of the composition of claim 1 as a vaccine to generate an immune response to Mycobacterium tuberculosis in a mammal.
